SEQUENCE LISTING (102 sequences)

SEQ ID NO: 1 - DNA, 372 bp, Homo sapiens
cagctgcagctgcaggagtcgggcccaggactggtgaagccttcggagaccctgtccctcacctgcactgtctctggtggctccatcaggagtagtagttactactggggctggatccgccagcccccagggaaggggctggagtggattgggggtatctattatagtgggagcacctactacaacccgtctctcaagagtcgagtcaccatgtccgtagacacgtccaagaaccagttctccctgaagctgagctccgtgaccgccgcagacacggctgtgtattactgtgcgagacaaaggggtcatagcagtggctggtggtacttcgatctctggggccgtggcaccctggtcactgtctcctcagcc

SEQ ID NO: 2 - protein, 124 aa, Homo sapiens
QLQLQESGPGLVKPSETLSLTCTVSGGSIRSSSYYWGWIRQPPGKGLEWIGGIYYSGSTYYNPSLKSRVTMSVDTSKNQFSLKLSSVTAADTAVYYCARQRGHSSGWWYFDLWGRGTLVTVSSA

SEQ ID NO: 3 - DNA, 324 bp, Homo sapiens
gacatccagatgacccagtctccatcttccgtgtctgcatctgtaggagacagtgtcaccatcacttgtcgggcgagtcagggtattagcagctacttagcctggtatcagcagaaaccagggaaagcccctaaactcctgatctatgctgcatccagtttgcaaagtggggtcccatcaaggttcagcggcaatggatctgggacagatttcactctcaccatcagcagcctgcagcctgaagattttgcaacttactattgtcaacaggctaacaatttcccattcactttcggccctgggaccaaagtggatatcaaacga

SEQ ID NO: 4 - protein, 108 aa, Homo sapiens
DIQMTQSPSSVSASVGDSVTITCRASQGISSYLAWYQQKPGKAPKLLIYAASSLQSGVPSRFSGNGSGTDFTLTISSLQPEDFATYYCQQANNFPFTFGPGTKVDIKR

SEQ ID NO: 5 - DNA, 363 bp, Homo sapiens
caggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagagacccttactactactactacggtatggacgtctggggccaagggaccacggtcaccgtctcctcagcc

SEQ ID NO: 6 - protein, 121 aa, Homo sapiens
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARDPYYYYYGMDVWGQGTTVTVSSA

SEQ ID NO: 7 - DNA, 336 bp, Homo sapiens
cagtctgtgttgacgcagccgccctcagtctctgcggccccaggacagaaggtcaccatctcctgctctggaagcagctccaacattgagaataatcatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcgaaacatgggataccagcctgagtgctggccgggtattcggcggagggaccaagctgaccgtcctaggt

SEQ ID NO: 8 - protein, 112 aa, Homo sapiens
QSVLTQPPSVSAAPGQKVTISCSGSSSNIENNHVSWYQQLPGTAPKLLIYDNNKRPSGIPDRFSGSKSGTSATLGITGLQTGDEADYYCETWDTSLSAGRVFGGGTKLTVLG

SEQ ID NO: 9 - DNA, 360 bp, Homo sapiens
caggtgcagctgcaggagtcgggcccaggactggtgaagccttcggagaccctgtccctcacctgcactgtctctggtggctccatcagtagttactactggagctggatccggcagcccccagggaggggactggagtggattggctatttcttttacagtgggtacaccaactacaacccctccctcaagagtcgcgtcaccatgtcagttgacacgtccaagaaccagttctctctgaagctgagctctgtgaccgctgcggacacggccgtgtattactgtgcgtgtataactggaacgacgaaggggggtatggacgtctggggccaaggggccacggtcaccgtctcctcagcc

SEQ ID NO: 10 - protein, 120 aa, Homo sapiens
QVQLQESGPGLVKPSETLSLTCTVSGGSISSYYWSWIRQPPGRGLEWIGYFFYSGYTNYNPSLKSRVTMSVDTSKNQFSLKLSSVTAADTAVYYCACITGTTKGGMDVWGQGATVTVSSA

SEQ ID NO: 11 - DNA, 336 bp, Homo sapiens
cagtctgtgctgacgcaggcgccctcagtgtctggggccccagggcagagggtcaccatctcctgcactgggagaagttccaacatcggggcaggttatgatgtacactggtaccagcagtttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagtctgagtggttcggtattcggcggagggaccaagctgaccgtcctaggt

SEQ ID NO: 12 - protein, 112 aa, Homo sapiens
QSVLTQAPSVSGAPGQRVTISCTGRSSNIGAGYDVHWYQQFPGTAPKLLIYGNSNRPSGVPDRFSGSKSGTSASLAITGLQAEDEADYYCQSYDSSLSGSVFGGGTKLTVLG

SEQ ID NO: 13 - DNA, 360 bp, Homo sapiens
caggtgcagctgcaggagtcgggcccaggactggtgaagccttcggagaccctgtccctcacctgcactgtctctggtggctccatcagtagttactactggagctggatccggcagcccccagggaagggactggagtggattgggtatttcttttacagtgggtacaccaactacaacccctccctcaagagtcgagtcaccatatcagtagacacgtccaagaaccagttctccctgaagctgagctctgtgaccgctgcggacacggccgtgtattactgtgcgtgtataactggaacgacgaaggggggtatggacgtctggggccaagggaccacggtcaccgtctcctcagcc

SEQ ID NO: 14 - protein, 120 aa, Homo sapiens
QVQLQESGPGLVKPSETLSLTCTVSGGSISSYYWSWIRQPPGKGLEWIGYFFYSGYTNYNPSLKSRVTISVDTSKNQFSLKLSSVTAADTAVYYCACITGTTKGGMDVWGQGTTVTVSSA

SEQ ID NO: 15 - DNA, 336 bp, Homo sapiens
cagtctgtactgacgcagccgccctcagtgtctggggccccagggcagagggtcaccatctcctgcactgggagcagctccaacatcggggcaggttatgatgtacactggtaccagcagcttccaggaacagcccccaagctcctcatctatggtaacaacaatcggccctcaggggtccctgaccgattctctggctccaagtctggcacctcagcctccctggccatcactgggctccaggctgatgatgaggctgattattactgccagtcctttgacagcagtctgagtggttcggtattcggcggagggaccaagctgaccgtcctaggt

SEQ ID NO: 16 - protein, 112 aa, Homo sapiens
QSVLTQPPSVSGAPGQRVTISCTGSSSNIGAGYDVHWYQQLPGTAPKLLIYGNNNRPSGVPDRFSGSKSGTSASLAITGLQADDEADYYCQSFDSSLSGSVFGGGTKLTVLG

SEQ ID NO: 17 - DNA, 372 bp, Homo sapiens
cagctgcagctgcaggagtcgggcccaggactggtgaagccttcggagaccctgtccctcacctgcactgtctctggtggctccatcaacagtagtagtaactactggggctggatccgccagcccccagggaagggactggcgtggattgggggcatctattatagtgggagcacctactacaacccgtccctcaggagtcgagtcaccatgtccgtagacacgtccaagaaccagttctccctgaagctgagctctgtgaccgccgcagacacggctgtatattactgtgcgagacaaaggggtcatagcagtggctggtggtacttcgatctctggggccgtggcaccctggtcactgtctcctcagcc

SEQ ID NO: 18 - protein, 124 aa, Homo sapiens
QLQLQESGPGLVKPSETLSLTCTVSGGSINSSSNYWGWIRQPPGKGLAWIGGIYYSGSTYYNPSLRSRVTMSVDTSKNQFSLKLSSVTAADTAVYYCARQRGHSSGWWYFDLWGRGTLVTVSSA

SEQ ID NO: 19 - DNA, 324 bp, Homo sapiens
gacatccagatgacccagtctccatcttccgtgtctgcatctgtaggagacagagtcaccatcacttgtcgggcgagtcggggtattagcagctggttagcctggtatcagcagagaccagggaaagcccctaagctcctgatctatactgcatccagtttgcaaagtggggtcccatcaaggttcagcggcagtggatctgggacagatttcactctcaccatcagcagcctgcagcctgaagattttgcaacttactattgtcaacaggctaacagtttcccattcactttcggccctgggaccaaagtggatatcaaacga

SEQ ID NO: 20 - protein, 108 aa, Homo sapiens
DIQMTQSPSSVSASVGDRVTITCRASRGISSWLAWYQQRPGKAPKLLIYTASSLQSGVPSRFSGSGSGTDFTLTISSLQPEDFATYYCQQANSFPFTFGPGTKVDIKR

SEQ ID NO: 21 - protein, 5 aa, Homo sapiens
SYYWS

SEQ ID NO: 22 - protein, 16 aa, Homo sapiens
YFFYSGYTNYNPSLKS

SEQ ID NO: 23 - protein, 11 aa, Homo sapiens
ITGTTKGGMDV

SEQ ID NO: 24 - protein, 14 aa, Homo sapiens
TGSSSNIGAGYDVH

SEQ ID NO: 25 - protein, 7 aa, Homo sapiens
GNNNRPS

SEQ ID NO: 26 - protein, 11 aa, Homo sapiens
QSFDSSLSGSV

SEQ ID NO: 27 - protein, 5 aa, Homo sapiens
SYYWS

SEQ ID NO: 28 - protein, 16 aa, Homo sapiens
YFFYSGYTNYNPSLKS

SEQ ID NO: 29 - protein, 11 aa, Homo sapiens
ITGTTKGGMDV

SEQ ID NO: 30 - protein, 14 aa, Homo sapiens
TGRSSNIGAGYDVH

SEQ ID NO: 31 - protein, 7 aa, Homo sapiens
GNSNRPS

SEQ ID NO: 32 - protein, 11 aa, Homo sapiens
QSYDSSLSGSV

SEQ ID NO: 33 - protein, 5 aa, Homo sapiens
SYDIN

SEQ ID NO: 34 - protein, 17 aa, Homo sapiens
WMNPNSGNTGYAQKFQG

SEQ ID NO: 35 - protein, 11 aa, Homo sapiens
DPYYYYYGMDV

SEQ ID NO: 36 - protein, 13 aa, Homo sapiens
SGSSSNIENNHVS

SEQ ID NO: 37 - protein, 7 aa, Homo sapiens
DNNKRPS

SEQ ID NO: 38 - protein, 12 aa, Homo sapiens
ETWDTSLSAGRV

SEQ ID NO: 39 - DNA, 594 bp, Homo sapiens
accatgaaacatctgtggttcttcctcctgctggtggcggctcccagatgggtcctgtcccagctgcagctgcaggagtcgggcccaggactggtgaagccttcggagaccctgtccctcacctgcactgtctctggtggctccatcaggagtagtagttactactggggctggatccgccagcccccagggaaggggctggagtggattgggggtatctattatagtgggagcacctactacaacccgtctctcaagagtcgagtcaccatgtccgtagacacgtccaagaaccagttctccctgaagctgagctccgtgaccgccgcagacacggctgtgtattactgtgcgagacaaaggggtcatagcagtggctggtggtacttcgatctctggggccgtggcaccctggtcactgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagct

SEQ ID NO: 40 - protein, 198 aa, Homo sapiens
TMKHLWFFLLLVAAPRWVLSQLQLQESGPGLVKPSETLSLTCTVSGGSIRSSSYYWGWIRQPPGKGLEWIGGIYYSGSTYYNPSLKSRVTMSVDTSKNQFSLKLSSVTAADTAVYYCARQRGHSSGWWYFDLWGRGTLVTVSSASTKGPSVFPLAPCSRSTSESTAALGCLVKDYFPEPVTVSWNSGALTSGVHTFPA

SEQ ID NO: 41 - DNA, 419 bp, Homo sapiens
atgagggtccctgctcagctcctggggctcctgctgctctggttcccaggttccagatgcgacatccagatgacccagtctccatcttccgtgtctgcatctgtaggagacagtgtcaccatcacttgtcgggcgagtcagggtattagcagctacttagcctggtatcagcagaaaccagggaaagcccctaaactcctgatctatgctgcatccagtttgcaaagtggggtcccatcaaggttcagcggcaatggatctgggacagatttcactctcaccatcagcagcctgcagcctgaagattttgcaacttactattgtcaacaggctaacaatttcccattcactttcggccctgggaccaaagtggatatcaaacgaactgtggctgcaccatctgtcttcatcttcccgcc

SEQ ID NO: 42 - protein, 139 aa, Homo sapiens
MRVPAQLLGLLLLWFPGSRCDIQMTQSPSSVSASVGDSVTITCRASQGISSYLAWYQQKPGKAPKLLIYAASSLQSGVPSRFSGNGSGTDFTLTISSLQPEDFATYYCQQANNFPFTFGPGTKVDIKRTVAAPSVFIFP

SEQ ID NO: 43 - DNA, 437 bp, Homo sapiens
accatggactggacctggaggatcctcttcttggtggcagcagctacaagtgcccactcccaggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagagacccttactactactactacggtatggacgtctggggccaagggaccacggtcaccgtctcctcagcctccaccaagggccc

SEQ ID NO: 44 - protein, 145 aa, Homo sapiens
TMDWTWRILFLVAAATSAHSQVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARDPYYYYYGMDVWGQGTTVTVSSASTKG

SEQ ID NO: 45 - DNA, 460 bp, Homo sapiens
atggcctggtctcctctcctcctcacccttctcattcactgcacagggtcctgggcccagtctgtgttgacgcagccgccctcagtctctgcggccccaggacagaaggtcaccatctcctgctctggaagcagctccaacattgagaataatcatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcgaaacatgggataccagcctgagtgctggccgggtattcggcggagggaccaagctgaccgtcctaggtcagcccaaggctgccccctcggtcactctgttcccaccctcctctgaggagctccaagccaacaagg

SEQ ID NO: 46 - protein, 153 aa, Homo sapiens
MAWSPLLLTLLIHCTGSWAQSVLTQPPSVSAAPGQKVTISCSGSSSNIENNHVSWYQQLPGTAPKLLIYDNNKRPSGIPDRFSGSKSGTSATLGITGLQTGDEADYYCETWDTSLSAGRVFGGGTKLTVLGQPKAAPSVTLFPPSSEELQANK

SEQ ID NO: 47 - DNA, 613 bp, Homo sapiens
accatgaaacatctgtggttcttccttctcctggtggcagctcccagatgggtcctgtcccaggtgcagctgcaggagtcgggcccaggactggtgaagccttcggagaccctgtccctcacctgcactgtctctggtggctccatcagtagttactactggagctggatccggcagcccccagggaggggactggagtggattggctatttcttttacagtgggtacaccaactacaacccctccctcaagagtcgcgtcaccatgtcagttgacacgtccaagaaccagttctctctgaagctgagctctgtgaccgctgcggacacggccgtgtattactgtgcgtgtataactggaacgacgaaggggggtatggacgtctggggccaaggggccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctca

SEQ ID NO: 48 - protein, 204 aa, Homo sapiens
TMKHLWFFLLLVAAPRWVLSQVQLQESGPGLVKPSETLSLTCTVSGGSISSYYWSWIRQPPGRGLEWIGYFFYSGYTNYNPSLKSRVTMSVDTSKNQFSLKLSSVTAADTAVYYCACITGTTKGGMDVWGQGATVTVSSASTKGPSVFPLAPCSRSTSESTAALGCLVKDYFPEPVTVSWNSGALTSGVHTFPAVLQSSGLYSL

SEQ ID NO: 49 - DNA, 432 bp, Homo sapiens
cctctgctcctcactctcctcgctcactgcacagggtcctgggcccagtctgtgctgacgcaggcgccctcagtgtctggggccccagggcagagggtcaccatctcctgcactgggagaagttccaacatcggggcaggttatgatgtacactggtaccagcagtttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagtctgagtggttcggtattcggcggagggaccaagctgaccgtcctaggtcagcccaaggctgccccctcggtcactctgttcccgccctcctctgaggag

SEQ ID NO: 50 - protein, 144 aa, Homo sapiens
PLLLTLLAHCTGSWAQSVLTQAPSVSGAPGQRVTISCTGRSSNIGAGYDVHWYQQFPGTAPKLLIYGNSNRPSGVPDRFSGSKSGTSASLAITGLQAEDEADYYCQSYDSSLSGSVFGGGTKLTVLGQPKAAPSVTLFPPSSEE

SEQ ID NO: 51 - DNA, 543 bp, Homo sapiens
atgaagcatctgtggttcttccttctcctggtggcagctcccagatgggtcctgtcccaggtgcagctgcaggagtcgggcccaggactggtgaagccttcggagaccctgtccctcacctgcactgtctctggtggctccatcagtagttactactggagctggatccggcagcccccagggaagggactggagtggattgggtatttcttttacagtgggtacaccaactacaacccctccctcaagagtcgagtcaccatatcagtagacacgtccaagaaccagttctccctgaagctgagctctgtgaccgctgcggacacggccgtgtattactgtgcgtgtataactggaacgacgaaggggggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactca

SEQ ID NO: 52 - protein, 181 aa, Homo sapiens
MKHLWFFLLLVAAPRWVLSQVQLQESGPGLVKPSETLSLTCTVSGGSISSYYWSWIRQPPGKGLEWIGYFFYSGYTNYNPSLKSRVTISVDTSKNQFSLKLSSVTAADTAVYYCACITGTTKGGMDVWGQGTTVTVSSASTKGPSVFPLAPCSRSTSESTAALGCLVKDYFPEPVTVSWNS

SEQ ID NO: 53 - DNA, 451 bp, Homo sapiens
tcctctgctcctcactctcctcgctcactgcacagggtcctgggcccagtctgtactgacgcagccgccctcagtgtctggggccccagggcagagggtcaccatctcctgcactgggagcagctccaacatcggggcaggttatgatgtacactggtaccagcagcttccaggaacagcccccaagctcctcatctatggtaacaacaatcggccctcaggggtccctgaccgattctctggctccaagtctggcacctcagcctccctggccatcactgggctccaggctgatgatgaggctgattattactgccagtcctttgacagcagtctgagtggttcggtattcggcggagggaccaagctgaccgtcctaggtcagcccaaggctgccccctcggtcactctgttcccgccctcctctgaggagctccaagccaacaaggaa

SEQ ID NO: 54 - protein, 145 aa, Homo sapiens
PLLLTLLAHCTGSWAQSVLTQPPSVSGAPGQRVTISCTGSSSNIGAGYDVHWYQQLPGTAPKLLIYGNNNRPSGVPDRFSGSKSGTSASLAITGLQADDEADYYCQSFDSSLSGSVFGGGTKLTVLGQPKAAPSVTLFPPSSEEL

SEQ ID NO: 55 - DNA, 559 bp, Homo sapiens
accatgaaacatctgtggttcttcctcctgctggtggcggctcccagatgggtcctgtcccagctgcagctgcaggagtcgggcccaggactggtgaagccttcggagaccctgtccctcacctgcactgtctctggtggctccatcaacagtagtagtaactactggggctggatccgccagcccccagggaagggactggcgtggattgggggcatctattatagtgggagcacctactacaacccgtccctcaggagtcgagtcaccatgtccgtagacacgtccaagaaccagttctccctgaagctgagctctgtgaccgccgcagacacggctgtatattactgtgcgagacaaaggggtcatagcagtggctggtggtacttcgatctctggggccgtggcaccctggtcactgtctcctcagcctccaccaagggcccatcggtcttccccctggcgccctgctccaggagcacctccgagagcacagcggccctgggctgcctggtcaaggactacttccccgaaccggtgacggtgtcgtggaactcag

SEQ ID NO: 56 - protein, 186 aa, Homo sapiens
TMKHLWFFLLLVAAPRWVLSQLQLQESGPGLVKPSETLSLTCTVSGGSINSSSNYWGWIRQPPGKGLAWIGGIYYSGSTYYNPSLRSRVTMSVDTSKNQFSLKLSSVTAADTAVYYCARQRGHSSGWWYFDLWGRGTLVTVSSASTKGPSVFPLAPCSRSTSESTAALGCLVKDYFPEPVTVSWNS

SEQ ID NO: 57 - DNA, 532 bp, Homo sapiens
atgagggtccccgctcagctcctggggctcctgctgctctggttcccaggttccagatgcgacatccagatgacccagtctccatcttccgtgtctgcatctgtaggagacagagtcaccatcacttgtcgggcgagtcggggtattagcagctggttagcctggtatcagcagagaccagggaaagcccctaagctcctgatctatactgcatccagtttgcaaagtggggtcccatcaaggttcagcggcagtggatctgggacagatttcactctcaccatcagcagcctgcagcctgaagattttgcaacttactattgtcaacaggctaacagtttcccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggta

SEQ ID NO: 58 - protein, 177 aa, Homo sapiens
MRVPAQLLGLLLLWFPGSRCDIQMTQSPSSVSASVGDRVTITCRASRGISSWLAWYQQRPGKAPKLLIYTASSLQSGVPSRFSGSGSGTDFTLTISSLQPEDFATYYCQQANSFPFTFGPGTKVDIKRTVAAPSVFIFPPSDEQLKSGTASVVCLLNNFYPREAKVQWKVDNALQSG

SEQ ID NO: 59 - DNA, 594 bp, Homo sapiens
accatgaaacatctgtggttcttcctcctgctggtggcggctcccagatgggtcctgtcccagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggggtatctattatagtgggagcacctactacaacccgtctctcaagagtcgagtcatcatgtccgtagacacgtccaagaaccagttctccctgaagctgagctccgtgaccgccgcagacacggctgtgtattactgtgcgagacaaaggggtcatagcagtggctggtggtacttcgatctctggggccgtggcaccctggtcactgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagct

SEQ ID NO: 60 - protein, 198 aa, Homo sapiens
TMKHLWFFLLLVAAPRWVLSQLQLQESGPGLVKPSETLSLTCTVSGGSISSSSYYWGWIRQPPGKGLEWIGGIYYSGSTYYNPSLKSRVTISVDTSKNQFSLKLSSVTAADTAVYYCARQRGHSSGWWYFDLWGRGTLVTVSSASTKGPSVFPLAPCSRSTSESTAALGCLVKDYFPEPVTVSWNSGALTSGVHTFPA

SEQ ID NO: 61 - DNA, 419 bp, Homo sapiens
atgagggtccctgctcagctcctggggctcctgctgctctggttcccaggttccagatgcgacatccagatgacccagtctccatcttccgtgtctgcatctgtaggagacagagtcaccatcacttgtcgggcgagtcagggtattagcagctacttagcctggtatcagcagaaaccagggaaagcccctaaactcctgatctatgctgcatccagtttgcaaagtggggtcccatcaaggttcagcggcagtggatctgggacagatttcactctcaccatcagcagcctgcagcctgaagattttgcaacttactattgtcaacaggctaacaatttcccattcactttcggccctgggaccaaagtggatatcaaacgaactgtggctgcaccatctgtcttcatcttcccgcc

SEQ ID NO: 62 - protein, 139 aa, Homo sapiens
MRVPAQLLGLLLLWFPGSRCDIQMTQSPSSVSASVGDRVTITCRASQGISSYLAWYQQKPGKAPKLLIYAASSLQSGVPSRFSGSGSGTDFTLTISSLQPEDFATYYCQQANNFPFTFGPGTKVDIKRTVAAPSVFIFP

SEQ ID NO: 63 - DNA, 437 bp, Homo sapiens
accatggactggacctggaggatcctcttcttggtggcagcagctacaagtgcccactcccaggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccagggacacctccataagcacagcctacatggagctgagcagcctgagatctgaggacacggccgtgtattactgtgcgagagacccttactactactactacggtatggacgtctggggccaagggaccacggtcaccgtctcctcagcctccaccaagggccc

SEQ ID NO: 64 - protein, 145 aa, Homo sapiens
TMDWTWRILFLVAAATSAHSQVQLVQSGAEVKKPGASVKVSCKASGYTFTSYDINWVRQATGQGLEWMGWMNPNSGNTGYAQKFQGRVTMTRDTSISTAYMELSSLRSEDTAVYYCARDPYYYYYGMDVWGQGTTVTVSSASTKG

SEQ ID NO: 65 - DNA, 460 bp, Homo sapiens; identical to SEQ ID NO: 45

SEQ ID NO: 66 - protein, 153 aa, Homo sapiens; identical to SEQ ID NO: 46

SEQ ID NO: 67 - DNA, 613 bp, Homo sapiens
accatgaaacatctgtggttcttccttctcctggtggcagctcccagatgggtcctgtcccaggtgcagctgcaggagtcgggcccaggactggtgaagccttcggagaccctgtccctcacctgcactgtctctggtggctccatcagtagttactactggagctggatccggcagcccccagggaagggactggagtggattggctatttcttttacagtgggtacaccaactacaacccctccctcaagagtcgcgtcaccatctcagttgacacgtccaagaaccagttctctctgaagctgagctctgtgaccgctgcggacacggccgtgtattactgtgcgcgtataactggaacgacgaaggggggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctca

SEQ ID NO: 68 - protein, 204 aa, Homo sapiens
TMKHLWFFLLLVAAPRWVLSQVQLQESGPGLVKPSETLSLTCTVSGGSISSYYWSWIRQPPGKGLEWIGYFFYSGYTNYNPSLKSRVTISVDTSKNQFSLKLSSVTAADTAVYYCARITGTTKGGMDVWGQGTTVTVSSASTKGPSVFPLAPCSRSTSESTAALGCLVKDYFPEPVTVSWNSGALTSGVHTFPAVLQSSGLYSL

SEQ ID NO: 69 - DNA, 432 bp, Homo sapiens
cctctgctcctcactctcctcgctcactgcacagggtcctgggcccagtctgtgctgacgcagccgccctcagtgtctggggccccagggcagagggtcaccatctcctgcactgggagaagttccaacatcggggcaggttatgatgtacactggtaccagcagttgccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagtctgagtggttcggtattcggcggagggaccaagctgaccgtcctaggtcagcccaaggctgccccctcggtcactctgttcccgccctcctctgaggag

SEQ ID NO: 70 - protein, 144 aa, Homo sapiens
PLLLTLLAHCTGSWAQSVLTQPPSVSGAPGQRVTISCTGRSSNIGAGYDVHWYQQLPGTAPKLLIYGNSNRPSGVPDRFSGSKSGTSASLAITGLQAEDEADYYCQSYDSSLSGSVFGGGTKLTVLGQPKAAPSVTLFPPSSEE

SEQ ID NO: 71 - DNA, 543 bp, Homo sapiens
atgaagcatctgtggttcttccttctcctggtggcagctcccagatgggtcctgtcccaggtgcagctgcaggagtcgggcccaggactggtgaagccttcggagaccctgtccctcacctgcactgtctctggtggctccatcagtagttactactggagctggatccggcagcccccagggaagggactggagtggattgggtatttcttttacagtgggtacaccaactacaacccctccctcaagagtcgagtcaccatatcagtagacacgtccaagaaccagttctccctgaagctgagctctgtgaccgctgcggacacggccgtgtattactgtgcgcgtataactggaacgacgaaggggggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactca

SEQ ID NO: 72 - protein, 181 aa, Homo sapiens
MKHLWFFLLLVAAPRWVLSQVQLQESGPGLVKPSETLSLTCTVSGGSISSYYWSWIRQPPGKGLEWIGYFFYSGYTNYNPSLKSRVTISVDTSKNQFSLKLSSVTAADTAVYYCARITGTTKGGMDVWGQGTTVTVSSASTKGPSVFPLAPCSRSTSESTAALGCLVKDYFPEPVTVSWNS

SEQ ID NO: 73 - DNA, 451 bp, Homo sapiens
tcctctgctcctcactctcctcgctcactgcacagggtcctgggcccagtctgtactgacgcagccgccctcagtgtctggggccccagggcagagggtcaccatctcctgcactgggagcagctccaacatcggggcaggttatgatgtacactggtaccagcagcttccaggaacagcccccaagctcctcatctatggtaacaacaatcggccctcaggggtccctgaccgattctctggctccaagtctggcacctcagcctccctggccatcactgggctccaggctgaagatgaggctgattattactgccagtcctttgacagcagtctgagtggttcggtattcggcggagggaccaagctgaccgtcctaggtcagcccaaggctgccccctcggtcactctgttcccgccctcctctgaggagctccaagccaacaaggaa

SEQ ID NO: 74 - protein, 145 aa, Homo sapiens
PLLLTLLAHCTGSWAQSVLTQPPSVSGAPGQRVTISCTGSSSNIGAGYDVHWYQQLPGTAPKLLIYGNNNRPSGVPDRFSGSKSGTSASLAITGLQAEDEADYYCQSFDSSLSGSVFGGGTKLTVLGQPKAAPSVTLFPPSSEEL

SEQ ID NO: 75 - protein, 119 aa, Homo sapiens
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARYYYYYGMDVWGQGTTVTVSSA

SEQ ID NO: 76 - protein, 110 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVFGGGTKLTVLG

SEQ ID NO: 77 - protein, 118 aa, Homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARSSWYFDLWGRGTLVTVSSA

SEQ ID NO: 78 - protein, 108 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPFTFGPGTKVDIKR

SEQ ID NO: 79 - protein, 117 aa, Homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARITGTGMDVWGQGTTVTVSSA

SEQ ID NO: 80 - protein, 112 aa, Homo sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGGGTKLTVLG

SEQ ID NO: 81 - protein, 7 aa, Homo sapiens
SSSYYWG

SEQ ID NO: 82 - protein, 16 aa, Homo sapiens
GIYYSGSTYYNPSLKS

SEQ ID NO: 83 - protein, 13 aa, Homo sapiens
QRGHSSGWWYFDL

SEQ ID NO: 84 - protein, 11 aa, Homo sapiens
RASQGISSYLA

SEQ ID NO: 85 - protein, 7 aa, Homo sapiens
AASSLQS

SEQ ID NO: 86 - protein, 9 aa, Homo sapiens
QQANNFPFT

SEQ ID NO: 87 - protein, 7 aa, Homo sapiens
SSSNYWG

SEQ ID NO: 88 - protein, 16 aa, Homo sapiens
GIYYSGSTYYNPSLRS

SEQ ID NO: 89 - protein, 13 aa, Homo sapiens
QRGHSSGWWYFDL

SEQ ID NO: 90 - protein, 11 aa, Homo sapiens
RASRGISSWLA

SEQ ID NO: 91 - protein, 7 aa, Homo sapiens
TASSLQS

SEQ ID NO: 92 - protein, 9 aa, Homo sapiens
QQANSFPFT

SEQ ID NO: 93 - protein, 25 aa, Homo sapiens [sequence omitted]

SEQ ID NO: 94 - protein, 10 aa, Homo sapiens
GYTFTSYDIN

SEQ ID NO: 95 - protein, 25 aa, Homo sapiens
QLQLQESGPGLVKPSETLSLTCTVS

SEQ ID NO: 96 - protein, 12 aa, Homo sapiens
GGSIRSSSYYWG

SEQ ID NO: 97 - protein, 25 aa, Homo sapiens
QLQLQESGPGLVKPSETLSLTCTVS

SEQ ID NO: 98 - protein, 12 aa, Homo sapiens
GGSINSSSNYWG

SEQ ID NO: 99 - protein, 25 aa, Homo sapiens
QVQLQESGPGLVKPSETLSLTCTVS

SEQ ID NO: 100 - protein, 10 aa, Homo sapiens
GGSISSYYWS

SEQ ID NO: 101 - protein, 25 aa, Homo sapiens
QVQLQESGPGLVKPSETLSLTCTVS

SEQ ID NO: 102 - protein, 10 aa, Homo sapiens
GGSISSYYWS

(19) United States
(12) Patent Application Publication
(10) Pub. No.: US 2013/0330353 A1
(43) Pub. Date: Dec. 12, 2013

(54) REGIMENS FOR TREATMENTS USING ANTI-IGF ANTIBODIES

(71) Applicants: Jaye Viner, Gaithersburg, MD (US); Yong Chang, Gaithersburg, MD (US); Jennifer McDevitt, Gaithersburg, MD (US)

(72) Inventors: Jaye Viner, Gaithersburg, MD (US); Yong Chang, Gaithersburg, MD (US); Jennifer McDevitt, Gaithersburg, MD (US)

(73) Assignee: MEDIMMUNE, LLC, Gaithersburg, MD (US)

(21) Appl. No.: 13/884,843

(22) PCT Filed: Nov. 15, 2011
(86) PCT No.: PCT/US11/60839

§ 371 Date: Aug. 23, 2013

Related U.S. Application Data
(60) Provisional application No. 61/414,318, filed on Nov. 16, 2010.
(60) Provisional application No. 61/529,614, filed on Aug. 31, 2011.

(51) Int. Cl. A61K 39/395

(52) U.S. Cl. CPC A61K 39/3955
USPC 424/145.1; 424/158.1

(57) ABSTRACT

Without limitation, this disclosure relates to methods of treating cell proliferation disorders, neoplastic disorders, cancers, tumors and the like using anti-IGF antibodies, or antigen binding fragments thereof. Disclosed herein are methods of treating cancer in a patient, for example a human patient, comprising administering to the patient at least two doses of an antibody which binds both IGF-I and/or IGF-II. The doses are separated by about a week, or by about three weeks, and each dose comprises an amount of antibody greater than about 0.5 mg kg of patient body mass and less than about 50 mg per kg of patient body mass.

### CROSS REFERENCE TO RELATED APPLICATIONS

This application is an international application and claims priority to U.S. Provisional Application No. 61/414,318, filed Nov. 16, 2010, and U.S. Provisional Application No. 61/529,614, filed Aug. 31, 2011, each of which is incorporated by reference in its entirety.

### BACKGROUND

1. Field

Without limitation, this disclosure relates to methods of treating cell proliferation disorders, neoplastic disorders, cancers, tumors and the like using anti-IGF antibodies and antigen binding fragments thereof.

2. Description of the Related Art

The insulin-like growth factor (IGF) system consists of ligands (IGF-I and IGF-II), the cell surface receptors (IGF-1R and IGF-2R), and the IGF-binding proteins (IGFBPs), all of which play a critical role in normal growth and development (Ryan P. D. et al. Oncologist. 2008; 13(1):16-24; Sachdev D. et al. Mol Cancer Ther. 2007; 6(1):1-12). IGF-I and IGF-II are small polypeptides involved in regulating cell proliferation, survival, differentiation, and transformation. Both are expressed ubiquitously and act as endocrine, paracrine, or autocrine growth factors. IGF-I and IGF-II exert their actions through binding to the IGF-I receptor tyrosine kinase (IGF-1R) and activate various intracellular signaling cascades. Activation of IGF signaling cascades leads to both stimulation of cell growth through activation of mitogen-activated protein kinase (MAPK) pathways as well as inhibition of apoptosis through stimulation of the protein kinase B (Akt) pathway. Insulin-like growth factors can also stimulate signaling through the insulin receptor (IR) pathway. There are two isoforms of the insulin receptor, IR-A and IR-B, which differ in the extra 12 amino acid residues present at the C-terminal end of the α-subunit of IR-B. Insulin receptor-B is the isoform that signals metabolic activities of insulin, while IR-A acts as a growth stimulatory signal, and is often overexpressed in tumor tissue compared to normal tissue. IGF-I and IGF-II can bind to a heterodimeric IGF-1R/IR receptor, and IGF-II can bind to homomeric IR receptors with affinities approaching insulin. Thus, IGFs can activate growth stimulatory signals through activating either IGF-1R or IR-A pathways. The binding properties of IGFs also suggest that inhibition of the IGF-1R receptor alone may incompletely inhibit IGF growth stimulatory activity. IGFs circulate in serum mostly bound to IGFBP-1 to 6. The interaction of IGFs with the IGF-1R is regulated by the IGFBPs, and IGFs can only bind to the IGF-1R once released from the IGFBPs. This release occurs mostly by proteolysis of the IGFBPs. Thus inhibition of “free” IGF is likely to result in a reduction of signal flux through the relevant receptors.

Numerous previously published preclinical studies have reported that down-regulation of IGF-1R expression and/or inhibition of signaling lead to inhibition of tumor growth, both in vitro and in vivo (Yuen J. S. et al. Expert Opin. Ther. Targets. 2008; 12(5):589-603). Inhibition of IGF signaling has also been shown to increase the susceptibility of tumor cells to chemotherapeutic agents (Wu K. D. et al. Cancer Immunol Immunother. 2007; 56(3):343-57). A variety of strategies (antisense oligonucleotides, soluble receptor, inhibitory peptides, dominant negative receptor mutants, small molecules that inhibit the kinase activity, and anti-hIGF-1R antibodies) have been developed to inhibit the IGF-1R signaling pathway in tumor cells. Each of these strategies has demonstrated the IGF-1R signaling pathway plays an important role in tumor cell growth and survival (Sachdev and Yee, 2007, supra).

In addition, epidemiologic studies support the assertion that IGFs play an important role in human cancers (Renehan A. G. et al. Lancet. 2004; 363(9418):1346-53; Wolpin B. M. et al. Cancer Res. 2007; 67(16):7923-8). High levels of circulating IGF-I are associated with an increased risk for development of several common cancers (Renehan et al., 2004, supra). In particular, the association is strongest for breast, prostate, and colorectal cancer but also present in non-small cell lung cancer (NSCLC), hepatocellular carcinoma (HCC), gastro-esophageal cancer, pancreatic cancer, and others.

In one prospective case-control study of prostate cancer, individuals with the highest quartile of IGF-I levels had a five-fold greater risk of advanced prostate cancer compared to those in the lowest quartile (Chan J. M. et al. Science. 1998; 279(5350):563-6). Similarly, levels of the principal binding protein of IGF (IGFBP-3), which acts as a negative regulator of IGF signaling, have a negative predictive value for development of many common cancers (Chan J. M. et al. J Natl Cancer Inst. 2002; 94(14):1099-106; Lu L. et al. Clin Cancer Res. 2006; 12(4):1208-14; Renehan et al., 2004, supra).

IGF signaling likely plays an important role in the development and/or progression of breast cancer. Epidemiologic studies suggest that elevated levels of IGF-I in serum correlate with a higher risk for developing breast cancer in women over the age of 50 (Rinaldi S. et al. Endocr Relat Cancer. 2006 June; 13(2):593-605). The IGF signaling cascade appears to be activated in numerous cancer types as determined by examination of human tumors. For example, IGF-1R levels are elevated in breast cancer cell lines and often in fresh tumor biopsies. Insulin-like growth factor 2 is expressed by both tumor and stromal cells, and IGF-I by stromal cells (Yee D. et al. Mol Endocrinol. 1989; 3(3):509-17). Insulin receptor is also often overexpressed in breast cancer, and it has recently been demonstrated that IR-A is the predominant insulin receptor isoform expressed in breast cancer cells (Sciacca L. et al. Oncogene. 1999; 18(15):2471-9). Cells that overexpress IR-A respond to treatment with IGF-II by growth stimulation (Sciacca et al, 1999, supra), suggesting a role for IGF-II in tumor growth through activation of IR signaling pathways. IGF signaling may have particular importance in relationship to the generation of resistance to effective anticancer therapies (Nahta R. et al. Nat Clin Pract Oncol. 2006; 3(5):269-80). Specifically, inhibition of IGF signaling has been shown to restore the growth inhibitory activity of trastuzumab in cells that had become resistant to HER2 blockade (Lu Y. et al. Biochem Biophys Res Commun. 2004a 313(3):709-15). Similarly, resistance to anti-estrogen therapy such as tamoxifen may be mediated in part by up-regulation of IGF signaling (Milano A. et al. Eur J Cancer. 2006; 42(16):2692-705).

In colon cancer, multiple lines of evidence suggest the importance of IGF signaling. Higher levels of IGF-I were found in patients with adenomas and advanced adenomas compared to controls without lesions (Schoen R. E. et al. Gastroenterology. 2005; 129(2):464-75). High serum IGF-II concentrations have also been found in patients with colorectal cancer, with a trend towards higher concentrations in advanced disease (Giovannucci E. J Nutr. 2001; 131(11 Suppl):3109S-20S). Additionally, most primary tumors and transformed cell lines overexpress IGF-II mRNA and protein. Overexpression of IGF-II in colon cancer is associated with an aggressive phenotype, and the loss of imprinting (loss of allele-specific expression) of the IGF-II gene may lead to higher expression and may be important in colorectal carcinogenesis (Woodson K. et al. J Natl Cancer Inst. 2004; 96(5):407-10). Cancer cells with a strong tendency to metastasize have significantly higher levels of IGF-II expression than those cells with a low ability to metastasize (Sekharam M. et al. Cancer Res. 2003; 63(22):7708-16). Insulin receptor-A is also reported to be expressed more frequently than IR-B in colon cancer (Frasca F. et al. Mol Cell Biol. 1999 May; 19(5):3278-88).

IGF-I, IGF-II, and IGF-1R are also overexpressed in bladder cancer (Zhao H et al. J Urol. 2003; 169:714-17; Gallagher E. M. et al. Hum Can Biol. 2008 14(21):6829-6838; Rochester M. A. et al. BJU Int 2007; 100:1396-1401). In vitro studies of human bladder cell lines demonstrate that IGF-I induces cell proliferation and blocks apoptosis (Iwamura M. et al. Urol Res 1993; 21:27-32). Furthermore, neutralization of IGF-1R signaling sensitizes urothelial cells to mitomycin-induced apoptosis (Sun H. Z. et al. Cell Res. 2001 June; 11(2):107-15). In vivo studies have shown that caloric restriction markedly reduces carcinogen-induced bladder cancers by lowering circulating IGF-I, an effect that is reversed by the administration of human IGF-I (Dunn S. E. et al. Cancer Res 1997; 57:4667-4672).

In the clinical setting, higher circulating levels of free IGF-I and IGF-II bound to its carrier IGFBP-3 have been found in patients with bladder cancer as compared to matched controls. In one study, mean IGF-I was significantly higher (175.8 versus 153.2 ng/ml, p<0.01) and mean IGFBP-3 was significantly lower (2,632.9 versus 3,056.6 ng/ml, p<0.01) in 154 cases as compared to 154 matched controls (Zhao H. et al. J Urol. 2003; 169:714-17). This study also found a significant association between the highest quartile plasma levels of IGF-I and risk of bladder cancer (odds ratio [OR] 3.10, 95% CI 1.43 to 6.70). Conversely, the highest quartile plasma levels of IGFBP-3 were associated with a reduced risk of bladder cancer (OR 0.38, 95% CI 0.19 to 0.78) (Zhao et al, 2003, supra).

IGF-II, a maternally imprinted fetal growth factor gene, regulates cellular proliferation and differentiation. Although the paternal chromosome typically expresses IGF-II while the imprinted maternal chromosome remains “silent”, in the setting of IGF-II loss of imprinting (LOI) bi-allelic expression of IGF-II has been associated with increased IGF-II protein levels, cellular hyperproliferation, and a broad array of solid, embryonal, and hematologic malignancies (Cui H. Disease Markers 2007; 23:105-12; Gallagher E. M. et al. Hum Can Biol. 2008 14(21):6829-6838). Beckwith-Wiedemann Syndrome (BWS), a genetic model for IGF-II LOI that is characterized by congenital overgrowth syndrome, is associated with embryonal tumors early in life such as nephroblastoma (also known as Wilms tumor), hepatoblastoma, neuroblastoma, adrenocortical carcinoma, and rhabdomyosarcoma.

Antibodies that bind IGF-I and IGF-II have been described. See, for example, Goya et al., Cancer Res. 64:6252-6258 (2004); Miyamoto et al., Clin. Cancer Res. 11:3494-3502 (2005); International Patent Application Publications WO2005/018671, WO2005/028515, and WO2003/093317; and U.S. Pat. No. 7,939,637. These publications also mention using antibodies that bind IGF-I and IGF-II to treat cancers, but they do not provide pharmacodynamic and pharmacokinetic data in humans to support a particular treatment regimen for use in humans. In particular, it is known in the art that providing too high a dose of a therapeutic compound can be deleterious to health, while providing too low a dose will not provide meaningful therapeutic benefit. Pharmacodynamic (PD) and pharmacokinetic (PK) data in humans provides the information for selecting a dosing regimen between these extremes. In addition, PD and PD data permit a dosing regimen designed to modulate a target of the antibody to a particular degree. For example, PK and PD data identify a dose of antibody that results in a particular concentration of antibody over a time interval that is sufficient to neutralize a target of the antibody by a specific amount in a human. When combined with data showing therapeutic activity in humans, the PK and PD data permit the identification of dosing regimens for neutralizing the target of the antibody to the degree sufficient for therapeutic benefit.

In the absence of PK and PD data from studies in humans, WO2005/018671, WO2005/028515, and WO2003/093317 suggest a wide range of antibody doses, from 10 μg per kg to 10 mg per kg per day for an adult human. Similarly, the U.S. Pat. No. 7,939,637 suggests doses for pancreatic cancer ranging from 50 mg per kg to 2,250 mg per kg for 4-8 weeks and suggests daily doses from about 0.001 mg per kg up to 100 mg per kg or more. These publications do not identify a dosing regimen that permits sustained concentrations of antibody in the circulation or a dosing regimen sufficient to neutralize IGF-I and/or IGF-II in a human. Similarly, these publications fail to provide any evidence that a particular dosing regimen, or level of neutralization of IGF-I and/or IGF-II, supports a therapeutic benefit. There is, therefore, a need for a treatment regimen using an antibody that binds IGF-I and IGF-II wherein the dose and administration schedule is based upon data in humans.

### SUMMARY

Previous suggested dose and administration schedules provide a broad range of possible doses for an antibody that binds IGF-I and IGF-II, but such dose and administration schedules are not based on data in humans. The behavior of an antibody in non-human animals, however, may not be predictive of its behavior in humans. Moreover in the case of the antibodies of the disclosure, which bind IGF-I and IGF-II, and can have different affinities for IGF-I and IGF-II, such modeling is particularly challenging. For example, it is challenging to predict the dose of antibody in humans that will suppress both ligands in serum or in tumors. In addition, non-human animal studies cannot identify a dose of antibody sufficient to neutralize antibody targets to a level that does not produce unacceptable toxicities. Disclosed herein are methods of treating cancer and symptoms resulting from IGF-I/II induced cell proliferation, and other diseases or conditions in which IGF affects the course or symptoms thereof, in a patient, for example a human patient, comprising administering to the patient at least two doses of an antibody, or an antigen binding fragment thereof, which binds IGF-I and IGF-II. In some examples, the doses are separated by about a week. In some examples of the method, the treatment comprises at least one cycle of three doses administered about once a week for three weeks. Alternatively, a treatment regimen can comprise administering at least two of said doses, separate doses being administered about three weeks apart.

Also disclosed herein are methods of suppressing IGF-I and/or IGF-II in the blood/and or in a tumor of a patient, comprising administering to the patient at least two doses of an antibody, or an antigen binding fragment thereof, which binds IGF-I and IGF-II wherein the doses are separated by about a week. In some examples of the method, the treatment comprises at least one cycle of three doses administered about once a week for three weeks. Alternatively, a treatment regimen can comprise administering at least two of said doses, separate doses being administered about three weeks apart.

In various examples, each dose can comprise about 0.5 mg per kg of body mass administered about once a week, to about 45 mg per kg of body mass administered about every three weeks. In one example the dose is less than about 50 mg per kg of body mass. In another example, each dose may comprise about 0.5 mg per kg of body mass administered about once a week to about 15 mg per kg of body mass administered about once a week. In another example, each dose may comprise about 1.5 mg per kg of body mass administered about once week to about 5 mg per kg of body mass administered about once a week. A dose may comprise at least 1.5 mg per kg of body mass administered about once a week. A dose may comprise about 5 mg per kg of body mass administered about once a week to about 15 mg per kg of body mass administered about once per week. In particular examples, a dose may contain about 0.5 mg per kg, 1.5 mg per kg, 5 mg per kg, 10 mg per kg, 15 mg per kg, administered about once a week. In particular examples, a dose may contain about 30 mg per kg or about 45 mg per kg, administered about every three weeks. In some examples, the cumulative three week dose may be between about 30 mg per kg or about 45 mg per kg. In another example, a treatment regimen may comprise administering one or more loading doses followed by one or more maintenance doses, where the loading doses are at least about two times greater than said maintenance doses. In other examples, one or more weeks may separate the doses (e.g., a dose every three weeks with a week in between successive three-week doses; a dose every three weeks, with a week between successive three week doses, dosing every other week).

In various examples, the amount of antibody, or antigen binding fragment thereof, which binds IGF-I and IGF-II in each dose may be selected so as to be sufficient to neutralize IGF-I and/or IGF-II in the patient's blood and/or tumor. In some examples, the amount of antibody, or antigen binding fragment thereof, which binds IGF-I and IGF-II may be selected to be sufficient to neutralize IGF-I and/or IGF-II in the patient's blood and/or tumor for at least about one day. In some examples, the amount of antibody, or antigen binding fragment thereof, which binds IGF-I and IGF-II may be selected to be sufficient to neutralize IGF-I and/or IGF-II in the patient's blood and/or tumor for at least about one week. Alternatively, the amount of antibody, or antigen binding fragment thereof, which binds IGF-I and IGF-II in each dose is sufficient to neutralize IGF-I and/or IGF-II in the patient's blood and/or tumor for about three weeks.

In some examples, the antibodies, or antigen binding fragments thereof, neutralize IGF-I in samples from treated subjects by greater than about 40% relative to biological samples from untreated subjects. In some examples, the antibodies, or antigen binding fragments thereof, neutralize IGF-II by greater than about 29% relative to biological samples from untreated subjects. In some examples, the antibodies of the disclosure neutralize IGF-I and/or IGF-II in biological samples from treated subjects in the range of about 70% to about 99% relative to biological samples from untreated subjects. In some examples, the antibodies of the disclosure neutralize IGF-I and/or IGF-II in biological samples by about 70%, 75%, 80%, 85%, 90%, 95% or 99% relative to biological samples from untreated subjects.

In some examples, the antibodies, or antigen binding fragments thereof, neutralize IGF-I in samples from treated subjects by greater than about 40% relative to biological samples from the subject prior to treatment. In some examples, the antibodies, or antigen binding fragments thereof, neutralize IGF-II by greater than about 29% relative to biological samples from the subject prior to treatment. In some examples, the antibodies of the disclosure neutralize IGF-I and/or IGF-II in biological samples from treated subjects in the range of about 70% to about 99% relative to biological samples from the subject prior to treatment. In some examples, the antibodies of the disclosure neutralize IGF-I and/or IGF-II in biological samples by 75%, 80%, 85%, 90%, 95% or 99% relative to biological samples from the subject prior to treatment. Biological samples may include blood and tumor tissue samples.

The methods of the disclosure include dosing regimens using antibodies, or antigen binding fragments thereof, that bind with greater affinity to IGF-II than IGF-I. In some examples, an antibody, or antigen binding fragment thereof, that binds to IGF-I/II can preferentially bind to IGF-II, but would cross-react with IGF-I, binding to IGF-II with higher affinity than to IGF-I. In some examples, an anti-IGF-I/II antibody, or antigen binding fragment thereof, binds to IGF-II with 2.5 times greater affinity than to IGF-I. In certain examples, the antibody, or antigen binding fragment thereof, can bind to IGF-II with at least 5, at least 10, at least 25, at least 50 or at least 150 times greater affinity than to IGF-I. In some examples, the antibody, or antigen binding fragment thereof, binds with at least 150 times greater affinity to IGF-II than to IGF-I.

In some examples, the antibody is chosen from mAb 7.251.3 (ATCC Accession Number PTA-7422), mAb 7.34.1 (ATCC Accession Number PTA-7423), and mAb 7.159.2 (ATCC Accession Number PTA-7424). In some examples, the antibody, or antigen binding fragment thereof, comprises a variable chain of mAb 7.251.3 (ATCC Accession Number PTA-7422), mAb 7.34.1 (ATCC Accession Number PTA-7423), and mAb 7.159.2 (ATCC Accession Number PTA-7424). In some examples, the antibody, or antigen binding fragment thereof, comprises a CDR chosen from mAb 7.251.3 (ATCC Accession Number PTA-7422), mAb 7.34.1 (ATCC Accession Number PTA-7423), and mAb 7.159.2 (ATCC Accession Number PTA-7424). In some examples, the antibody, or antigen binding fragment thereof, comprises at least one variable chain polypeptide having an amino acid sequence chosen from SEQ ID NOs: 2, 6, 10, 14, 18, 40, 44, 48, 52, 56, 60, 64, 68, and 72. In some examples, the antibody, or antigen binding fragment thereof, comprises at least one variable chain polypeptide having an amino acid sequence chosen from SEQ ID NOs: 4, 8, 12, 16, 20, 42, 46, 50, 54, 58, 62, 66, 70, 74. In some examples, the antibody, or antigen binding fragment thereof, comprises at least one variable chain polypeptide having an amino acid sequence chosen from SEQ ID NOs: 2, 6, 10, 14, 18, 40, 44, 48, 52, 56, 60, 64, 68, and 72, and at least one variable chain polypeptide having an amino acid sequence chosen from SEQ ID NOs: 4, 8, 12, 16, 20, 42, 46, 50, 54, 58, 62, 66, 70, 74. In some examples, the antibody comprises a heavy chain comprising three CDRs chosen from the CDRs shown in Table 2. In some examples, the antibody comprises a light chain comprising three CDRs chosen from the CDRs shown in Table 3. In some examples, the antibody comprises a heavy chain comprising three CDRs chosen from the CDRs shown in Table 2 and a light chain comprising three CDRs chosen from the CDRs shown in Table 3. In some examples, the CDRs comprise the CDRs of mAb 7.251.3. In some examples, the CDRs comprise the CDRs of mAb 7.34.1. In some examples, the CDRs comprise the CDRs of mAb 7.159.2.

In some examples, the antibody, or antigen binding fragment thereof, is administered at a dose providing a Cmax from about 12 to about 560 μg/ml. In some examples, the antibody or antigen binding fragment thereof, is administered at a dose providing a Cmax from about 12 to about 588 μg/ml. In some examples, the antibody, or antigen binding fragment thereof, is administered at a dose providing an AUC from about 6 to about 1940 μg*d/ml. In some examples, the antibody, or antigen binding fragment thereof, is administered at a dose providing an AUC from about 6 to about 3620 μg*d/ml.

In some examples, diseases of the methods include cancers. In some examples the cancers are melanoma, non-small cell lung cancer, glioma, hepatocellular (liver) carcinoma, thyroid tumor, gastric (stomach) cancer, prostate cancer, breast cancer, ovarian cancer, bladder cancer, lung cancer, brain cancer including glioblastoma, uterine cancer, endometrial cancer, kidney cancer, colon cancer, gynecologic tumors, head and neck cancer, esophageal cancer, and pancreatic cancer and sarcoma such as epidermoid carcinoma, Ewing's sarcoma, angiosarcoma, and liposarcoma. In some examples, the cancer is chosen from breast, hepatocellular, or bladder cancer. In some examples, the foregoing cancers are primary cancers. In some examples, the forgoing cancers are metastatic cancers.

### BRIEF DESCRIPTION OF THE DRAWINGS

FIG. 1A-D show inhibition of C32 tumor growth in mice by MEDI-573 at different doses and administration schedules.

FIGS. 2 A and B show serum concentration-time profiles for free IGF-I and IGF-II (as Percent Change From Baseline) following administration of MEDI-573 at 1, 3, 10, and 30 mg/kg on days 1 and 8 in cynomolgus monkeys.

FIG. 3 shows pharmacokinetic results at time points following administration of MEDI-573 to patients in a 3+3 dose escalation study in human subjects.

FIGS. 4 A and B show levels of IGF-I and IGF-II in patient's circulation at time points following administration of MEDI-573 in a 3+3 dose escalation study in human subjects. For illustration purposes, BQL (below quantitative limit) samples were plotted as ½ LLOQ (lower limit of quantitation) level. Panel B shows the levels of IGF-I and IGF-II in patient's circulation for seven days after administration of MEDI-573.

FIG. 5 shows MEDI-573 treatment exposure and activity. The black bars indicate the seven patients that showed disease stabilization (bladder cancer, liposarcoma, angiosarcoma, Ewing's sarcoma, uterine cancer, rectal cancer, and prostate cancer), and the two patients remaining on study treatment (bladder cancer and liposarcoma) are indicated by stars.

### DETAILED DESCRIPTION

Disclosed herein are methods of treating cancer and symptoms resulting from IGF-I/II mediated cell proliferation, and other diseases or conditions in which IGF affects the course or symptoms thereof, in a patient, for example a human patient, comprising administering to the patient at least two doses of an antibody, or antigen binding fragment thereof, which binds IGF-I and IGF-II, wherein the doses are separated by about a week and each dose comprises about 0.5 mg per kg of patient body mass to about 15 mg per kg of body mass, with the total dose over a three week period ranging from about 1.5 mg per kg of patient body mass to about 45 mg of per kg of patient body mass.

In some examples of the method, the treatment comprises at least one cycle of three doses administered about once a week for three weeks. Alternatively, a treatment regimen can comprise administering at least two of said doses, separate doses being administered about three weeks apart.

Also disclosed herein are methods of neutralizing IGF-I and/or IGF-II in the blood/and tumor of a patient, comprising administering to the patient of at least two doses of an antibody, or antigen binding fragment thereof, which binds IGF-I and IGF-II, wherein the doses are separated by about a week and each dose comprises about 0.5 mg per kg of patient body mass to about 15 mg per kg of body mass, with the total dose over a three week period ranging from about 1.5 mg per kg of patient body mass to about 45 mg per kg of patient body mass. In some examples of the methods, the treatment comprises at least one cycle of three doses administered about once a week for three weeks. Alternatively, a treatment regimen can comprise administering at least two of said doses, separate doses being administered about three weeks apart.

The methods described herein may be used to treat diseases or conditions in which IGF affects the course or symptoms thereof, including neoplastic diseases, such as, melanoma, non-small cell lung cancer, glioma, hepatocellular (liver) carcinoma, thyroid tumor, gastric (stomach) cancer, prostate cancer, breast cancer, ovarian cancer, bladder cancer, lung cancer, brain cancer including glioblastoma, endometrial cancer, kidney cancer, colon cancer, gynecologic tumors, head and neck cancer, esophageal cancer, and pancreatic cancer and sarcoma such as epidermoid carcinoma, Ewing's sarcoma, angiosarcoma, and liposarcoma. In particular examples of cancers that can be treated, the method disclosed herein can be used to treat bladder cancer, breast cancer, and/or hepatocellular carcinoma. The cancers treated with the disclosed methods may be primary cancers or metastatic cancers.

The amount of antibody, or antigen binding fragment thereof, which binds IGF-I and IGF-II in each dose may be selected so as to be sufficient to neutralize IGF-I and/or IGF-II in the patient's blood. In some examples, the amount of antibody, or antigen binding fragment thereof, which binds IGF-I and IGF-II may be selected to be sufficient to neutralize IGF-I and/or IGF-II in the patient's blood for at least one day. In some examples, the amount of antibody, or antigen binding fragment thereof, which binds IGF-I and IGF-II may be selected to be sufficient to neutralize IGF-I and/or IGF-II in the patient's blood for at least about one week. Alternatively, the amount of antibody, or antigen binding fragment thereof, which binds IGF-I and IGF-II in each dose may be sufficient to neutralize IGF-I and/or IGF-II in the patient's blood for about three weeks.

In some examples, the antibodies, or antigen binding fragments thereof, of the disclosure neutralize IGF-I and/or IGF-II in biological samples from treated subjects in the range of about 70% to about 99%, or completely neutralize, relative to biological samples from untreated subjects. In some examples, the antibodies, or antigen binding fragments thereof, of the disclosure neutralize IGF-I and/or IGF-II in biological samples by 70%, 75%, 80%, 85%, 90%, 95%, 99 or 100% relative to biological samples from untreated subjects. In some examples, the antibodies, or antigen binding fragments thereof, of the disclosure neutralize IGF-I and/or IGF-II in biological samples from treated subjects in the range of 70% to 99%, or completely neutralize, relative to biological samples from the subject prior to treatment. In some examples, the antibodies, or antigen binding fragments thereof, of the disclosure neutralize IGF-I and/or IGF-II in biological samples by 75%, 80%, 85%, 90%, 95%, 99% or 100% relative to biological samples from the subject prior to treatment.

In various examples, each dose of antibody, or antigen binding fragment thereof, can comprise about 0.5 mg per kg of body mass administered about once a week to about 45 mg per kg of body mass administered about every three weeks. Weekly dosing may comprise about 0.5 mg per kg of body mass to about 15 mg per kg of body mass. Weekly dosing may comprise about 0.5 mg per kg of body mass to about 5 mg per kg of body mass. A dose may comprise about 5 mg per kg of body mass to about 15 mg per kg of body mass. In particular examples, a dose may contain about 0.5 mg per kg, 1.5 mg per kg, 5 mg per kg, 10 mg per kg, 15 mg per kg, 30 mg per kg, or 45 mg per kg. In additional examples, the dose may be about 0.5 mg per kg, 1.5 mg per kg, 5 mg per kg, 10 mg per kg, or 15 mg per kg administered about once a week. In another example, the dose may be about 30 mg per kg, or 45 mg per kg about every three weeks. Each dose may be separated by one or more periods of non-dosing. In one example, a dose administered about every three weeks is followed by a week in which no dose is administered. In another example, a dose is administered every other week. In another example, a dose is administered every week for about three weeks, followed by a week in which no dose is administered. In another example, a treatment regimen may comprise administering one or more loading doses followed by one or more maintenance doses, where the loading doses are at least about two times greater than said maintenance doses.

In some examples, the anti-IGF-I/II antibody, or antigen binding fragment thereof, binds to IGF-II with 2.5 times greater affinity than to IGF-I. In certain examples, the antibody or antigen binding fragment thereof, binds to IGF-II with at least 5, at least 10, at least 25, at least 50 or at least 150 times greater affinity than to IGF-I.

In some examples the antibody is a fully human monoclonal antibody, and is chosen from mAb 7.251.3 (ATCC Accession Number PTA-7422), mAb 7.34.1 (ATCC Accession Number PTA-7423), and mAb 7.159.2 (ATCC Accession Number PTA-7424). In some examples, the antibody, or antigen binding fragment thereof, comprises a variable chain of mAb 7.251.3 (ATCC Accession Number PTA-7422), mAb 7.34.1 (ATCC Accession Number PTA-7423), and mAb 7.159.2 (ATCC Accession Number PTA-7424). In some examples, the antibody, or antigen binding fragment thereof, comprises a CDR chosen from mAb 7.251.3 (ATCC Accession Number PTA-7422), mAb 7.34.1 (ATCC Accession Number PTA-7423), and mAb 7.159.2 (ATCC Accession Number PTA-7424). In some examples, the antibody, or antigen binding fragment thereof, comprises a heavy chain comprising 3 CDRs of from mAb 7.251.3 (ATCC Accession Number PTA-7422), mAb 7.34.1 (ATCC Accession Number PTA-7423), and mAb 7.159.2 (ATCC Accession Number PTA-7424), and a light chain. In some examples, the antibody, or antigen binding fragment thereof, comprises a light chain comprising 3 CDRs of from mAb 7.251.3 (ATCC Accession Number PTA-7422), mAb 7.34.1 (ATCC Accession Number PTA-7423), and mAb 7.159.2 (ATCC Accession Number PTA-7424), and a heavy chain. In some examples, the antibody or antigen binding fragment thereof, comprises 6 CDRs chosen mAb 7.251.3 (ATCC Accession Number PTA-7422), mAb 7.34.1 (ATCC Accession Number PTA-7423), and mAb 7.159.2 (ATCC Accession Number PTA-7424). In some examples, the antibody, or antigen binding fragment thereof, comprises the six CDRs of mAb 7.251.3 (ATCC Accession Number PTA-7422). In some examples, the antibody or antigen binding fragment comprises the six CDRs of mAb 7.34.1 (ATCC Accession Number PTA-7423). In some examples, the antibody or antigen binding fragment comprises the six CDRs of mAb 7.159.2 (ATCC Accession Number PTA-7424).

### DEFINITIONS

The term “IGF-I” refers to the molecule Insulin-like growth factor-I, and the term “IGF-II” refers to the molecule Insulin-like growth factor-II. The term “IGF-I/II” refers to both insulin-like growth factors-I and -II.

The terms “neutralize” and “neutralizing” when referring to an antibody or antigen binding fragment thereof relates to the ability of an antibody to eliminate, or reduce, the activity of target antigen. The term also refers to reducing the amount of the target antigen. Accordingly, a “neutralizing” anti-IGF-I, anti-IGF-II, or anti-IGF-I/II antibody is capable of reducing or eliminating the activity or amount of free IGF-I and/or IGF-II. A neutralizing anti-IGF-I and anti-IGF-II antibody may, for example, act by blocking the binding of IGF-I and/or IGF-II to its receptor IGF-1R or IR-A. By blocking this binding, the IGF-1R mediated signal transduction is significantly, or completely, eliminated. Alternatively, a neutralizing antibody may, for example, reduce or eliminate the amount of free IGF-I and/or II in the blood and/or in a tumor, thus, or example, reducing or eliminating free IGF-I and/or IGF-II available for binding to receptor. When the term “suppress” or “suppression” is used in the context of IGF-I and IGF-II, it has the same meaning as “neutralize” and “neutralizing.”

As used herein, the term “antibody” refers to a polypeptide or group of polypeptides that are comprised of at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions, or variable chain polypeptides, of each light/heavy chain pair form an antibody binding site. The term “mAb” refers to monoclonal antibody.

In some examples, an antibody, or antigen binding fragment thereof, that binds to IGF-I/II can preferentially bind to IGF-II, but would cross-react with IGF-I, binding to IGF-II with higher affinity than to IGF-I. For example, an anti-IGF-I/II antibody, or antigen binding fragment thereof, might bind to IGF-II with 2.5 times greater affinity than to IGF-I. In certain examples, the antibody, or antigen binding fragment thereof, can bind to IGF-II with at least 5, at least 10, at least 25, at least 50 or at least 150 times greater affinity than to IGF-I.

“Binding fragments” of an antibody are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)₂ fragment in which the two arms of the antibody molecule remain linked and comprise two-antigen binding sites. The F(ab′)₂ fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CH1 domain of the heavy chain.

The term “agent” is used herein to denote a chemical compound, a mixture of chemical compounds, a biological macromolecule, or an extract made from biological materials.

“Active” or “activity” in regard to an IGF-I/II polypeptide refers to a portion of an IGF-I/II polypeptide that has a biological or an immunological activity of a native IGF-I/II polypeptide. “Biological” when used herein refers to a biological function that results from the activity of the native IGF-I/II polypeptide. A particular IGF-I/II biological activity includes, for example, IGF-I/II induced cell proliferation.

“Mammal” when used herein refers to any animal that is considered a mammal. Preferably, the mammal is human.

“Liposome” when used herein refers to a small vesicle that may be useful for delivery of drugs that may include the antibodies of the disclosure.

“Label” or “labeled” as used herein refers to the addition of a detectable moiety to a polypeptide, for example, a radiolabel, fluorescent label, enzymatic label chemiluminescent labeled or a biotinyl group. Radioisotopes or radionuclides may include ³H, ¹⁴C, ¹⁵N, ³⁵S, ⁹⁰Y, ⁹⁹Tc, ¹¹¹In, ¹²⁵I, ¹³¹I, fluorescent labels may include rhodamine, lanthanide phosphors or FITC and enzymatic labels may include horseradish peroxidase, β-galactosidase, luciferase, alkaline phosphatase.

The term “pharmaceutical agent or drug” as used herein refers to a chemical compound or composition capable of inducing a desired therapeutic effect when properly administered to a patient.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

Treating and treatment as used herein refer to procedures which are effective to cure or reduce a symptom of, cause a regression of, slow the progression of, stop the progression of, and/or which may be combined with another procedure to improve the treatment of, a disease or condition such as cancer. It is understood that treatments may not always provide a cure. Thus, a successful treatment may prolong the survival of a patient or alleviate an undesirable symptom.

A dose refers to a single administration of a therapeutic composition. Dosage refers to the amount of a therapeutically active molecule in a dose. A treatment regimen refers to the dosage, schedule, and mode of administration of one or more doses. A cycle refers to a repeatable unit of one or more doses within a treatment regimen. In some treatment regimens dosages are uniform for each dose. In other treatment regimens, the dosages may not be uniform. For example, one or more loading doses may be used to raise the concentration of a therapeutic molecule to a desired level in a patient. Loading doses may be followed by one or more maintenance doses, generally comprising lower dosages (for example one half or less of a loading dose) which are sufficient to maintain a desired concentration of a therapeutic molecule in a patient. One or more tapering doses may be used to gradually reduce the concentration of a therapeutic molecule in a patient.

Patient refers to a subject, which may be a human or other mammal, in need of treatment for one or more diseases or conditions. The term “patient” includes human and veterinary subjects.

### Procedures for Making Human or Humanized Anti-IGF I/II Antibodies

Human antibodies avoid some of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, fully human antibodies can be generated through the introduction of functional human antibody loci into a rodent, other mammal or animal so that the rodent, other mammal or animal produces fully human antibodies.

One method for generating fully human antibodies is through the use of XenoMouse® strains of mice that have been engineered to contain up to but less than 1000 kb-sized germline configured fragments of the human heavy chain locus and kappa light chain locus. See Mendez et al. Nature Genetics 15:146-156 (1997) and Green and Jakobovits J. Exp. Med. 188:483-495 (1998). The XenoMouse® strains are available from Abgenix, Inc. (Fremont, Calif.).

The production of the XenoMouse® strains of mice is further discussed and delineated in U.S. patent application Ser. Nos. 07/466,008, filed Jan. 12, 1990, 07/610,515, filed Nov. 8, 1990, 07/919,297, filed Jul. 24, 1992, 07/922,649, filed Jul. 30, 1992, 08/031,801, filed Mar. 15, 1993, 08/112,848, filed Aug. 27, 1993, 08/234,145, filed Apr. 28, 1994, 08/376,279, filed Jan. 20, 1995, 08/430, 938, filed Apr. 27, 1995, 08/464,584, filed Jun. 5, 1995, 08/464,582, filed Jun. 5, 1995, 08/463,191, filed Jun. 5, 1995, 08/462,837, filed Jun. 5, 1995, 08/486,853, filed Jun. 5, 1995, 08/486,857, filed Jun. 5, 1995, 08/486,859, filed Jun. 5, 1995, 08/462,513, filed Jun. 5, 1995, 08/724,752, filed Oct. 2, 1996, 08/759,620, filed Dec. 3, 1996, U.S. Publication 2003/0093820, filed Nov. 30, 2001 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more V_{H} genes, one or more D_{H} genes, one or more J_{H} genes, a mu constant region, and usually a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023.010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, filed Aug. 29, 1990, 07/575,962, filed Aug. 31, 1990, 07/810,279, filed Dec. 17, 1991, 07/853,408, filed Mar. 18, 1992, 07/904,068, filed Jun. 23, 1992, 07/990,860, filed Dec. 16, 1992, 08/053,131, filed Apr. 26, 1993, 08/096,762, filed Jul. 22, 1993, 08/155,301, filed Nov. 18, 1993, 08/161,739, filed Dec. 3, 1993, 08/165,699, filed Dec. 10, 1993, 08/209,741, filed Mar. 9, 1994, the disclosures of which are hereby incorporated by reference. See also European Patent No. 0 546 073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

Kirin has also demonstrated the generation of human antibodies from mice in which, through microcell fusion, large pieces of chromosomes, or entire chromosomes, have been introduced. See European Patent Application Nos. 773 288 and 843 961, the disclosures of which are hereby incorporated by reference. Additionally, KM™—mice, which are the result of cross-breeding of Kirin's Tc mice with Medarex's minilocus (Humab) mice have been generated. These mice possess the human IgH transchromosome of the Kirin mice and the kappa chain transgene of the Genpharm mice (Ishida et al., Cloning Stem Cells, (2002) 4:91-102).

Human antibodies can also be derived by in vitro methods. Suitable examples include but are not limited to phage display (CAT, Morphosys, Dyax, Biosite/Medarex, Xoma, Symphogen, Alexion (formerly Proliferon), Affimed) ribosome display (CAT), yeast display, and the like.

Antibodies, as described herein, were prepared through the utilization of the XenoMouse® technology, as described below. Such mice, then, are capable of producing human immunoglobulin molecules and antibodies and are deficient in the production of murine immunoglobulin molecules and antibodies. Technologies utilized for achieving the same are disclosed in the patents, applications, and references disclosed in the background section herein. In particular, however, a preferred embodiment of transgenic production of mice and antibodies therefrom is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al. Nature Genetics 15:146-156 (1997), the disclosure of which is hereby incorporated by reference.

Through the use of such technology, fully human monoclonal antibodies to a variety of antigens have been produced. Essentially, XenoMouse® lines of mice are immunized with an antigen of interest (e.g. IGF-I/II), lymphatic cells (such as B-cells) are recovered from the hyper-immunized mice, and the recovered lymphocytes are fused with a myeloid-type cell line to prepare immortal hybridoma cell lines. These hybridoma cell lines are screened and selected to identify hybridoma cell lines that produced antibodies specific to the antigen of interest. Provided herein are methods for the production of multiple hybridoma cell lines that produce antibodies specific to IGF-I/II. Further, provided herein are characterization of the antibodies produced by such cell lines, including nucleotide and amino acid sequence analyses of the heavy and light chains of such antibodies.

Alternatively, instead of being fused to myeloma cells to generate hybridomas, B cells can be directly assayed. For example, CD19+ B cells can be isolated from hyperimmune XenoMouse® mice and allowed to proliferate and differentiate into antibody-secreting plasma cells. Antibodies from the cell supernatants are then screened by ELISA for reactivity against the IGF-I/II immunogen. The supernatants might also be screened for immunoreactivity against fragments of IGF-I/II to further map the different antibodies for binding to domains of functional interest on IGF-I/II. The antibodies may also be screened against other related human chemokines and against the rat, the mouse, and non-human primate, such as cynomolgus monkey, orthologues of IGF-I/II, the last to determine species cross-reactivity. B cells from wells containing antibodies of interest may be immortalized by various methods including fusion to make hybridomas either from individual or from pooled wells, or by infection with EBV or transfection by known immortalizing genes and then plating in suitable medium. Alternatively, single plasma cells secreting antibodies with the desired specificities are then isolated using an IGF-I/II-specific hemolytic plaque assay (Babcook et al., Proc. Natl. Acad. Sci. USA 93:7843-48 (1996)). Cells targeted for lysis are preferably sheep red blood cells (SRBCs) coated with the IGF-I/II antigen.

In the presence of a B-cell culture containing plasma cells secreting the immunoglobulin of interest and complement, the formation of a plaque indicates specific IGF-I/II-mediated lysis of the sheep red blood cells surrounding the plasma cell of interest. The single antigen-specific plasma cell in the center of the plaque can be isolated and the genetic information that encodes the specificity of the antibody is isolated from the single plasma cell. Using reverse-transcription followed by PCR (RT-PCR), the DNA encoding the heavy and light chain variable regions of the antibody can be cloned. Such cloned DNA can then be further inserted into a suitable expression vector, preferably a vector cassette such as a pcDNA, more preferably such a pcDNA vector containing the constant domains of immunglobulin heavy and light chain. The generated vector can then be transfected into host cells, e.g., HEK293 cells, CHO cells, and cultured in conventional nutrient media modified as appropriate for inducing transcription, selecting transformants, or amplifying the genes encoding the desired sequences.

In general, antibodies produced by the fused hybridomas were human IgG2 heavy chains with fully human kappa or lambda light chains. Antibodies described herein possess human IgG4 heavy chains as well as IgG2 heavy chains. Antibodies can also be of other human isotypes, including IgG1. The antibodies possessed high affinities, typically possessing a Kd of from about 10⁻⁶ through about 10⁻¹² M or below, when measured by solid phase and solution phase techniques. Antibodies possessing a KD of at least 10⁻¹¹ M are desired to inhibit the activity of IGF-I/II.

As will be appreciated, anti-IGF-I/II antibodies can be expressed in cell lines other than hybridoma cell lines. Sequences encoding particular antibodies can be used to transform a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introducing heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells, and a number of other cell lines. Cell lines of particular preference are selected through determining which cell lines have high expression levels and produce antibodies with constitutive IGF-I/II binding properties.

### Exemplary Antibodies

Anti-IGF antibodies suitable for use in the treatment methods described herein are described in Goya et al., Cancer Res. 64:6252-6258 (2004); Miyamoto et al., Clin. Cancer Res. 11:3494-3502 (2005); International Patent Application Publications WO2005/018671, WO2005/028515, and WO2003/093317; and U.S. Pat. No. 7,939,637. The descriptions of the anti-IGF antibodies disclosed therein are incorporated herein by reference.

Particular antibodies include those that are described in U.S. Pat. No. 7,939,637. These include the specific anti-IGF-I/II antibodies listed below in Table 1. This table reports the identification number of each anti-IGF-I/II antibody, along with the SEQ ID number of the corresponding heavy chain and light chain genes. Further, the germline sequences from which each heavy chain and light chain derive are also listed below in Table 1.

Each antibody has been given an identification number that includes either two or three numbers separated by one or two decimal points. In some cases, several clones of one antibody were prepared. Although the clones have the identical nucleic acid and amino acid sequences as the parent sequence, they may also be listed separately, with the clone number indicated by the number to the right of a second decimal point. Thus, for example, the nucleic acid and amino acid sequences of antibody 7.159.2 are identical to the sequences of antibody 7.159.1.

As can be seen by comparing the sequences in the sequence listing, SEQ ID NOs.: 1-20 differ from SEQ ID NOs.: 39-58 because SEQ ID NOs.: 39-58 include the untranslated, signal peptide, and constant domain regions for each sequenced heavy or light chain.

TABLE 1

| | | SEQ |
|---|---|---|
| mAb ID | | ID |
| No.: | Sequence | NO: |
| 7.158.1 | Nucleotide sequence encoding the variable region of the heavy chain | 1 |
| | Amino acid sequence encoding the variable region of the heavy chain | 2 |
| | Nucleotide sequence encoding the variable region of the light chain | 3 |
| | Amino acid sequence encoding the variable region of the light chain | 4 |
| 7.159.2 | Nucleotide sequence encoding the variable region of the heavy chain | 5 |
| | Amino acid sequence encoding the variable region of the heavy chain | 6 |
| | Nucleotide sequence encoding the variable region of the light chain | 7 |
| | Amino acid sequence encoding the variable region of the light chain | 8 |
| 7.34.1 | Nucleotide sequence encoding the variable region of the heavy chain | 9 |
| | Amino acid sequence encoding the variable region of the heavy chain | 10 |
| | Nucleotide sequence encoding the variable region of the light chain | 11 |
| | Amino acid sequence encoding the variable region of the light chain | 12 |
| 7.251.3 | Nucleotide sequence encoding the variable region of the heavy chain | 13 |
| | Amino acid sequence encoding the variable region of the heavy chain | 14 |
| | Nucleotide sequence encoding the variable region of the light chain | 15 |
| | Amino acid sequence encoding the variable region of the light chain | 16 |
| 7.234.1 | Nucleotide sequence encoding the variable region of the heavy chain | 17 |
| | Amino acid sequence encoding the variable region of the heavy chain | 18 |
| | Nucleotide sequence encoding the variable region of the light chain | 19 |
| | Amino acid sequence encoding the variable region of the light chain | 20 |
| 7.158.1 | Nucleotide sequence encoding the variable region of the heavy chain | 39 |
| | Amino acid sequence encoding the variable region of the heavy chain | 40 |
| | Nucleotide sequence encoding the variable region of the light chain | 41 |
| | Amino acid sequence encoding the variable region of the light chain | 42 |
| 7.159.2 | Nucleotide sequence encoding the variable region of the heavy chain | 43 |
| | Amino acid sequence encoding the variable region of the heavy chain | 44 |
| | Nucleotide sequence encoding the variable region of the light chain | 45 |
| | Amino acid sequence encoding the variable region of the light chain | 46 |
| 7.34.1 | Nucleotide sequence encoding the variable region of the heavy chain | 47 |
| | Amino acid sequence encoding the variable region of the heavy chain | 48 |
| | Nucleotide sequence encoding the variable region of the light chain | 49 |
| | Amino acid sequence encoding the variable region of the light chain | 50 |
| 7.251.3 | Nucleotide sequence encoding the variable region of the heavy chain | 51 |
| | Amino acid sequence encoding the variable region of the heavy chain | 52 |
| | Nucleotide sequence encoding the variable region of the light chain | 53 |
| | Amino acid sequence encoding the variable region of the light chain | 54 |
| 7.234.1 | Nucleotide sequence encoding the variable region of the heavy chain | 55 |
| | Amino acid sequence encoding the variable region of the heavy chain | 56 |
| | Nucleotide sequence encoding the variable region of the light chain | 57 |
| | Amino acid sequence encoding the variable region of the light chain | 58 |
| Germline | Nucleotide sequence encoding the variable region of the heavy chain | 59 |
| (7.158.1) | Amino acid sequence encoding the variable region of the heavy chain | 60 |
| | Nucleotide sequence encoding the variable region of the light chain | 61 |
| | Amino acid sequence encoding the variable region of the light chain | 62 |
| Germline | Nucleotide sequence encoding the variable region of the heavy chain | 63 |
| (7.159.1) | Amino acid sequence encoding the variable region of the heavy chain | 64 |
| | Nucleotide sequence encoding the variable region of the light chain | 65 |
| | Amino acid sequence encoding the variable region of the light chain | 66 |
| Germline | Nucleotide sequence encoding the variable region of the heavy chain | 67 |
| (7.34.1) | Amino acid sequence encoding the variable region of the heavy chain | 68 |
| | Nucleotide sequence encoding the variable region of the light chain | 69 |
| | Amino acid sequence encoding the variable region of the light chain | 70 |
| Germline | Nucleotide sequence encoding the variable region of the heavy chain | 71 |
| (7.251.3) | Amino acid sequence encoding the variable region of the heavy chain | 72 |
| | Nucleotide sequence encoding the variable region of the light chain | 73 |
| | Amino acid sequence encoding the variable region of the light chain | 74 |

The complete sequence information for the anti-IGF-I/II antibodies is provided in the sequence listing with nucleotide and amino acid sequences for each gamma and kappa chain combination. The variable heavy sequences were analyzed to determine the VH family, the D-region sequence and the J-region sequence. The sequences were then translated to determine the amino acid sequence and compared to the germline VH, D and J-region sequences to assess somatic hypermutations.

The alignment of the sequences of these antibodies to their germline genes are shown in the following tables. Table 2 is a table comparing the antibody heavy chain regions to their cognate germ line heavy chain region. Table 3 is a table comparing the antibody kappa light chain regions to their cognate germ line light chain region. Identity is shown as “-” and mutations away from germline are shown as the new amino acid.

The variable (V) regions of immunoglobulin chains are encoded by multiple germ line DNA segments, which are joined into functional variable regions (V_{H}DJ_{H} or V_{K}J_{K}) during B-cell ontogeny. The molecular and genetic diversity of the antibody response to IGF-I/II was studied in detail. These assays revealed several points specific to anti-IGF-I/II antibodies.

Analysis of five individual antibodies specific to IGF-I/II resulted in the determination that the antibodies were derived from three different germline VH genes, four of them from the VH4 family, with two antibodies being derived from the VH4-39 gene segment. Tables 2 and 3 show the results of this analysis.

It should be appreciated that amino acid sequences among the sister clones collected from each hybridoma are identical. For example, the heavy chain and light chain sequences for mAb 7.159.2 are identical to the sequences shown in Tables 2 and 3 for mAb 7.159.1.

The heavy chain CDR1s of the antibodies of the disclosure have a sequence as disclosed in Table 2. The CDR1s disclosed in Table 2 are of the Kabat definition. Alternatively, the CDR1s can be defined using an alternative definition so as to include the last five residues of the FR1 sequence. For example, for antibody 7.159.1 the FR1 sequence is QVQLVQSGAEVKKPGASVKVSCKAS (SEQ ID NO.: 93) and the CDR1 sequence is GYTFTSYDIN (SEQ ID NO.: 94); for antibody 7.158.1 the FR1 sequence is QLQLQESGPGLVKPSETLSLTCTVS (SEQ ID NO.: 95) and the CDR1 sequence is GGSIRSSSYYWG (SEQ ID NO.: 96); for antibody 7.234.1 the FR1 sequence is QLQLQESGPGLVKPSETLSLTCTVS (SEQ ID NO.: 97) and the CDR1 sequence is GGSINSSSNYWG (SEQ ID NO.: 98); for antibody 7.34.1 the FR1 sequence is QVQLQESGPGLVKPSETLSLTCTVS (SEQ ID NO.: 99) and the CDR1 sequence is GGSISSYYWS (SEQ ID NO.: 100); and for antibody 7.251.3 the FR1 sequence is QVQLQESGPGLVKPSETLSLTCTVS (SEQ ID NO.: 101) and the CDR1 sequence is GGSISSYYWS (SEQ ID NO.: 102).

It should also be appreciated that where a particular antibody differs from its respective germline sequence at the amino acid level, the antibody sequence can be mutated back to the germline sequence. Such corrective mutations can occur at one, two, three or more positions, or a combination of any of the mutated positions, using standard molecular biological techniques. By way of non-limiting example, Table 3 shows that the light chain sequence of mAb 7.34.1 (SEQ ID NO.: 12) differs from the corresponding germline sequence (SEQ ID NO.:80) through a Pro to Ala mutation (mutation 1) in the FR1 region, and via a Phe to Leu mutation (mutation 2) in the FR2 region. Thus, the amino acid or nucleotide sequence encoding the light chain of mAb 7.34.1 can be modified to change mutation 1 to yield the germline sequence at the site of mutation 1. Further, the amino acid or nucleotide sequence encoding the light chain of mAb 7.34.1 can be modified to change mutation 2 to yield the germline sequence at the site of mutation 2. Still further, the amino acid or nucleotide sequence encoding the light chain of mAb 7.34.1 can be modified to change both mutation 1 and mutation 2 to yield the germline sequence at the sites of both mutations 1 and 2.

TABLE 2
HEAVY CHAIN ANALYSIS

| | SEQ | | | | | | | | | | |
|---|---|---|---|---|---|---|---|---|---|---|---|
| Chain | ID | | | | | | | | | | |
| Name | NO. | V | D | J | FR1 | CDR1 | FR2 | CDR2 | FR3 | CDR3 | FR4 |
| Germline | 75 | VH1-8 | N.A. | JH6B | QVQLV | SYDIN | WVRQATG | WMNPNS | RVTMTRNTS | ##YYY | WGQG |
| | | | | | QSGAE | | QGLEWMG | GNTGYA | ISTAYMELS | YYGMD | TTVT |
| | | | | | VKKPG | | | QKFQG | SLRSEDTAV | V | VSSA |
| | | | | | ASVKV | | | | YYCAR | | |
| | | | | | SCKAS | | | | | | |
| | | | | | GYTFT | | | | | | |
| 7.159.1 | 6 | ″ | ″ | ″ | QVQLV | SYDIN | WVRQATG | WMNPNS | RVTMTRNTS | DPYYY | WGQG |
| | | | | | QSGAE | | QGLEWMG | GNTGYA | ISTAYMELS | YYGMD | TTVT |
| | | | | | VKKPG | | | QKFQG | SLRSEDTAV | V | VSSA |
| | | | | | ASVKV | | | | YYCAR | | |
| | | | | | SCKAS | | | | | | |
| | | | | | GYTFT | | | | | | |
| Germline | 77 | VH4-39 | D6-19 | JH2 | QLQLQ | SSSY | WIRQPPG | SIYYSG | RVTISVDTS | ####S | WGRG |
| | | | | | ESGPG | YWG | KGLEWIG | STYYNP | KNQFSLKLS | S##WY | TLVT |
| | | | | | LVKPS | | | SLKS | SVTAADTAV | FDL | VSSA |
| | | | | | ETLSL | | | | YYCAR | | |
| | | | | | TCTVS | | | | | | |
| | | | | | GGSIS | | | | | | |
| 7.158.1 | 2 | ″ | ″ | ″ | QLQLQ | SSSY | WIRQPPG | GIYYSG | RVTMSVDTS | QRGHS | WGRG |
| | | | | | ESGPG | YWG | KGLEWIG | STYYNP | KNQFSLKLS | SGWWY | TLVT |
| | | | | | LVKPS | | | SLKS | SVTAADTAV | FDL | VSSA |
| | | | | | ETLSL | | | | YYCAR | | |
| | | | | | TCTVS | | | | | | |
| | | | | | GGSIR | | | | | | |
| 7.234.1 | 18 | ″ | ″ | ″ | QLQLQ | SSSN | WIRQPPG | GIYYSG | RVTMSVDTS | QRGHS | WGRG |
| | | | | | ESGPG | YWG | KGLAWIG | STYYNP | KNQFSLKLS | SGWWY | TLVT |
| | | | | | LVKPS | | | SLRS | SVTAADTAV | FDL | VSSA |
| | | | | | ETLSL | | | | YYCAR | | |
| | | | | | TCTVS | | | | | | |
| | | | | | GGSIN | | | | | | |
| Germline | 79 | VH4-59 | D1-20 | JH6B | QVQLQ | SYYWS | WIRQPPG | YIYYSG | RVTISVDTS | ITGT# | WGQG |
| | | | | | ESGPG | | KGLEWIG | STNYNP | KNQFSLKLS | ##GMD | TTVT |
| | | | | | LVKPS | | | SLKS | SVTAADTAV | V | VSSA |
| | | | | | ETLSL | | | | YYCA#R | | |
| | | | | | TCTVS | | | | | | |
| | | | | | GGSIS | | | | | | |
| 7.34.1 | 10 | ″ | ″ | ″ | QVQLQ | SYYWS | WIRQPPG | YFFYSG | RVTMSVDTS | ITGTT | WGQG |
| | | | | | ESGPG | | RGLEWIG | YTNYNP | KNQFSLKLS | KGGMD | ATVT |
| | | | | | LVKPS | | | SLKS | SVTAADTAV | V | VSSA |
| | | | | | ETLSL | | | | YYCAC | | |
| | | | | | TCTVS | | | | | | |
| | | | | | GGSIS | | | | | | |
| 7.251.3 | 14 | ″ | ″ | ″ | QVQLQ | SYYWS | WIRQPPG | YFFYSG | RVTISVDTS | ITGTT | WGQG |
| | | | | | ESGPG | | KGLEWIG | YTNYNP | KNQFSLKLS | KGGMD | TTVT |
| | | | | | LVKPS | | | SLKS | SVTAADTAV | V | VSSA |
| | | | | | ETLSL | | | | YYCAC | | |
| | | | | | TCTVS | | | | | | |
| | | | | | GGSIS | | | | | | |

* The hatch designation (#) indicates a space in the germline and is used to show a proper alignment with the antibody sequences shown in the table.
** The germline sequences shown in the above table are for alignment purposes, and it should be realized that each individual antibody region exists in its own location within the variable regions of immunoglobulin germline DNA segments in vivo.

TABLE 3
LIGHT CHAIN ANALYSIS

| | SEQ | | | | | | | | | |
|---|---|---|---|---|---|---|---|---|---|---|
| Chain | ID | | | | | | | | | |
| Name | NO. | V | J | FR1 | CDR1 | FR2 | CDR2 | FR3 | CDR3 | FR4 |
| Germline | 76 | V1-19 | JL2 | QSVLTQ | SGSSSNI | WYQQL | DNNK | GIPDRFSG | GTWDS | FGGG |
| | | | | PPSVSA | GNNYVS | PGTAP | RPS | SKSGTSAT | SLSA# | TKLT |
| | | | | APGQKV | | KLLIY | | LGITGLQT | #V | VLG |
| | | | | TISC | | | | GDEADYYC | | |
| 7_159_1 | 8 | ″ | ″ | QSVLTQ | SGSSSNI | WYQQL | DNNK | GIPDRFSG | ETWDT | FGGG |
| | | | | PPSVSA | ENNHVS | PGTAP | RPS | SKSGTSAT | SLSAG | TKLT |
| | | | | APGQKV | | KLLIY | | LGITGLQT | RV | VLG |
| | | | | TISC | | | | GDEADYYC | | |
| Germline | 78 | L5 | JK3 | DIQMTQ | RASQGIS | WYQQK | AASS | GVPSRFSG | QQANS | FGPG |
| | | | | SPSSVS | SWLA | PGKAP | LQS | SGSGTDFT | FPFT | TKVD |
| | | | | ASVGDR | | KLLIY | | LTISSLQP | | IKR |
| | | | | VTITC | | | | EDFATYYC | | |
| 7_158_1 | 4 | ″ | ″ | DIQMTQ | RASQGIS | WYQQK | AASS | GVPSRFSG | QQANN | FGPG |
| | | | | SPSSVS | SYLA | PGKAP | LQS | NGSGTDFT | FPFT | TKVD |
| | | | | ASVGDS | | KLLIY | | LTISSLQP | | IKR |
| | | | | VTITC | | | | EDFATYYC | | |
| 7_234_1 | 20 | ″ | ″ | DIQMTQ | RASRGIS | WYQQR | TASS | GVPSRFSG | QQANS | FGPG |
| | | | | SPSSVS | SWLA | PGKAP | LQS | SGSGTDFT | FPFT | TKVD |
| | | | | ASVGDR | | KLLIY | | LTISSLQP | IKR | |
| | | | | VTITC | | | | EDFATYYC | | |
| Germline | 80 | V1-13 | JL2 | QSVLTQ | TGSSSNI | WYQQL | GNSN | GVPDRFSG | QSYDS | FGGG |
| | | | | PPSVSG | GAGYDVH | PGTAP | RPS | SKSGTSAS | SLSGS | TKLT |
| | | | | APGQRV | | KLLIY | | LAITGLQA | V | VLG |
| | | | | TISC | | | | EDEADYYC | | |
| 7_34_1 | 12 | ″ | ″ | QSVLTQ | TGRSSNI | WYQQF | GNSN | GVPDRFSG | QSYDS | FGGG |
| | | | | APSVSG | GAGYDVH | PGTAP | RPS | SKSGTSAS | SLSGS | TKLT |
| | | | | APGQRV | | KLLIY | | LAITGLQA | V | VLG |
| | | | | TISC | | | | EDEADYYC | | |
| 7_251_3 | 16 | ″ | ″ | QSVLTQ | TGSSSNI | WYQQL | GNNN | GVPDRFSG | QSFDS | FGGG |
| | | | | PPSVSG | GAGYDVH | PGTAP | RPS | SKSGTSAS | SLSGS | TKLT |
| | | | | APGQRV | | KLLIY | | LAITGLQA | V | VLG |
| | | | | TISC | | | | DDEADYYC | | |

* The hatch designation (#) indicates a space in the germline and is used to show a proper alignment with the antibody sequences shown in the table.
** The germline sequences shown in the above table are for alignment purposes, and it should be realized that each individual antibody region exists in its own location within the variable regions of immunoglobulin germline DNA segments in vivo.

A high resolution Biacore analysis has been performed to further measure the antibody affinity to the antigen, as described in U.S. Pat. No. 7,939,637. mAbs 7.159.1, 7.234.2, 7.34.1, 7.251.3, and 7.160.2 were each captured and the IGF-I and IGF-II antigens were each injected over a range of concentrations. The resulting binding constants are listed in the following table.

ANTI-IGF ANTIBODY AFFINITY DETERMINED BY LOW-AND
HIGH-RESOLUTION BIACORE ANALYSIS

| | |
|---|---|
| Low resolution | High Resolution |
| K_{D} (pM) | K_{D} (pM) |

| mAb | IGF-I | IGF-II | IGF-I | IGF-II |
|---|---|---|---|---|
| 7.159.1 | 216.0 | 2.9 | 294.0 | 1.9 |
| 7.234.2 | 328.0 | 45.3 | 3760.0 | 295.0 |
| 7.34.1 | 615.0 | 60.0 | 436.0 | 164.0 |
| | | | 421.0 | 162.0 |
| 7.251.3 | 935.0 | 123.0 | 452.0 | 47.4 |
| 7.160.2 | 589.0 | 54.3 | 2800.0 | 237.0 |

### Modes of Administration and Formulations

Sterile pharmaceutical formulations of anti-IGF-I/II antibodies are useful in the methods disclosed herein. Sterile formulations can be created, for example, by filtration through sterile filtration membranes, prior to or following lyophilization and reconstitution of the antibody. The antibody ordinarily will be stored in lyophilized form or in solution. Therapeutic antibody compositions generally are placed into a container having a sterile access port, for example, an intravenous solution bag or vial having an adapter that allows retrieval of the formulation, such as a stopper pierceable by a hypodermic injection needle.

The route of antibody administration is in accord with known methods, e.g., injection or infusion by intravenous, intraperitoneal, intracerebral, intramuscular, intraocular, intraarterial, intrathecal, inhalation or intralesional routes, or by sustained release systems as noted below. The antibody is can be administered continuously by infusion or by bolus injection.

In certain examples, a therapist may titer the dosage within the range of dosages described herein, as guided by the PK, PD, and efficacy data disclosed herein, and may modify the route of administration as required to obtain the optimal therapeutic effect.

Antibodies, as described herein, can be prepared in a mixture with a pharmaceutically acceptable carrier. This therapeutic composition can be administered intravenously or through the nose or lung, preferably as a liquid or powder aerosol (lyophilized). The composition may also be administered parenterally or subcutaneously as desired. When administered systemically, the therapeutic composition should be sterile, pyrogen-free and in a parenterally acceptable solution having due regard for pH, isotonicity, and stability. These conditions are known to those skilled in the art. Briefly, dosage formulations of the compounds described herein are prepared for storage or administration by mixing the compound having the desired degree of purity with physiologically acceptable carriers, excipients, or stabilizers. Such materials are non-toxic to the recipients at the dosages and concentrations employed, and include buffers such as TRIS HC1, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN, PLURONICS or polyethyleneglycol.

Sterile compositions for injection can be formulated according to conventional pharmaceutical practice as described in Remington: The Science and Practice of Pharmacy (20^{th} ed, Lippincott Williams & Wilkens Publishers (2003)). For example, dissolution or suspension of the active compound in a vehicle such as water or naturally occurring vegetable oil like sesame, peanut, or cottonseed oil or a synthetic fatty vehicle like ethyl oleate or the like may be desired. Buffers, preservatives, antioxidants and the like can be incorporated according to accepted pharmaceutical practice.

Suitable examples of sustained-release preparations include semipermeable matrices of solid hydrophobic polymers containing the polypeptide, which matrices are in the form of shaped articles, films or microcapsules. Examples of sustained-release matrices include polyesters, hydrogels (e.g., poly(2-hydroxyethyl-methacrylate) as described by Langer et al., J. Biomed Mater. Res., (1981) 15:167-277 and Langer, Chem. Tech., (1982) 12:98-105, or poly(vinylalcohol)), polylactides (U.S. Pat. No. 3,773,919, EP 58,481), copolymers of L-glutamic acid and gamma ethyl-L-glutamate (Sidman et al., Biopolymers, (1983) 22:547-556), non-degradable ethylene-vinyl acetate (Langer et al., supra), degradable lactic acid-glycolic acid copolymers such as the LUPRON Depot™ (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through disulfide interchange, stabilization may be achieved by modifying sulfhydryl residues, lyophilizing from acidic solutions, controlling moisture content, using appropriate additives, and developing specific polymer matrix compositions.

Sustained-released compositions also include preparations of crystals of the antibody suspended in suitable formulations capable of maintaining crystals in suspension. These preparations when injected subcutaneously or intraperitonealy can produce a sustained release effect. Other compositions also include liposomally entrapped antibodies. Liposomes containing such antibodies are prepared by methods known per se: U.S. Pat. No. DE 3,218,121; Epstein et al., Proc. Natl. Acad. Sci. USA, (1985) 82:3688-3692; Hwang et al., Proc. Natl. Acad. Sci. USA, (1980) 77:4030-4034; EP 52,322; EP 36,676; EP 88,046; EP 143,949; 142,641; Japanese patent application 83-118008; U.S. Pat. Nos. 4,485,045 and 4,544,545; and EP 102,324.

It will be appreciated that administration of therapeutic entities in accordance with the compositions and methods herein will be administered with suitable carriers, excipients, and other agents that are incorporated into formulations to provide improved transfer, delivery, tolerance, and the like. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present disclosure, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol. Pharmacol. 32(2):210-8 (2000), Wang W. Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

### Exemplary Methods

1. A method of treating cancer in a patient, said method comprising administering to the patient at least two doses of an antibody, or antigen binding fragment thereof, which binds IGF-I and IGF-II, wherein doses are separated by about a week, and wherein each dose is between about 1.5 mg per kg of body mass and about 15 mg per mg per kg of body mass.

2. The method of embodiment 1, wherein the administering comprises administering at least three of said doses for about three weeks.

3. A method of treating cancer in a patient, the method comprising administering to the patient at least two doses of an antibody, or antigen binding fragment thereof, which binds IGF-I and IGF-II, wherein doses are separated by about three weeks, and wherein each dose is between about 30 mg per kg of body mass and about 45 mg per mg per kg of body mass.

4. The method of any of embodiments 1-3, wherein each dose is sufficient to neutralize IGF-I by greater than about 40% and IGF-II by greater than about 29%.

5. The method of any of embodiments 1-4, wherein each dose is sufficient to neutralize IGF-I and IGF-II by greater than about 90%.

6. The method of any of embodiments 1-5, wherein each dose is sufficient to neutralize IGF-I and IGF-II for at least one day.

7. The method of any of embodiments 1-5, wherein each dose is sufficient to neutralize IGF-I and IGF-II for at least about one week.

8. The method of any of embodiments 3-5, wherein each dose is sufficient to neutralize IGF-I and IGF-II for at least about three weeks.

9. The method of any of embodiments 1-8, wherein IGF-I and IGF-II are neutralized in the blood of the patient.

10. The method of any of embodiments 1-8, wherein IGF-I and IGF-II are neutralized in a tumor of the patient.

11. The method any of embodiments 1, 2, and 4-10, wherein each said dose comprises about 1.5 mg per kg of body mass to about 15 mg per kg of body mass.

12. The method any of embodiments 1, 2, and 4-10, wherein each said dose comprises about 5 mg per kg of body mass.

13. The method any of embodiments 1, 2, and 4-10, wherein each said dose comprises about 10 mg per kg of body mass.

14. The method any of embodiments 1, 2, and 4-10, wherein each said dose comprises about 15 mg per kg of body mass.

15. The method of any of embodiments 3-10, wherein each dose comprises about 30 mg per kg of body mass.

16. The method of any of embodiments 3-10, wherein each dose comprises about 45 mg per kg of body mass.

17. The method of any of embodiments 1-16, wherein the administering comprises administering one or more loading doses followed by one or more maintenance doses, and wherein said loading doses are at least about two times greater than said maintenance doses.

18. The method of any of embodiments 1-17, wherein the cancer is a cancer of the breast, bladder, prostate, colon, uterus, rectum, throat, lung, a colorectal cancer, non-small cell lung cancer, a sarcoma, or hepatocellular carcinoma.

19. The method of embodiment 18, wherein the cancer is bladder cancer.

20. The method of embodiment 18, wherein the cancer is hepatocellular carcinoma.

21. The method of embodiment 18, wherein the cancer is breast cancer.

22. The method of embodiment 18, wherein the cancer is a sarcoma.

23. The method of embodiment 18, wherein the cancer is prostate cancer.

24. The method of embodiment 18, wherein the cancer is rectal cancer.

25. The method of any of embodiments 18-24, wherein the cancer is a primary tumor cancer.

26. The method of any of embodiments 18-24, wherein the tumor cancer is a metastatic tumor cancer.

27. The method of any of embodiments 1-26, wherein the antibody which binds IGF-I and IGF-II is selected from among mAb 7.251.3, mAb 7.34.1, and mAb 7.159.2.

28. The method of embodiment 27, wherein the antibody is mAb 7.251.3.

29. The method of embodiment 27, wherein the antibody is mAb 7.34.1.

30. The method of embodiment 27, wherein the antibody is mAb 7.159.2.

31. The method of any of embodiments 1-26, wherein the antibody, or antigen binding fragment thereof, comprises at least one variable chain polypeptide having an amino acid sequence chosen from SEQ ID NOs: 2, 6, 10, 14, 18, 40, 44, 48, 52, 56, 60, 64, 68, and 72.

32. The method of any of embodiments 1-26, wherein the antibody, or antigen binding fragment thereof, comprises at least one variable chain polypeptide having an amino acid sequence chosen from SEQ ID NOs: 4, 8, 12, 16, 20, 42, 46, 50, 54, 58, 62, 66, 70, 74.

33. The method of any of embodiments 1-26, wherein the antibody, or antigen binding fragment thereof, comprises at least one variable chain polypeptide having an amino acid sequence chosen from SEQ ID NOs: 2, 6, 10, 14, 18, 40, 44, 48, 52, 56, 60, 64, 68, and 72, and at least one variable chain polypeptide having an amino acid sequence chosen from SEQ ID NOs: 4, 8, 12, 16, 20, 42, 46, 50, 54, 58, 62, 66, 70, 74.

34. The method of any of embodiments 1-26, wherein the antibody, or antigen binding fragment thereof, comprises a heavy chain comprising three CDRs chosen from the CDRs shown in Table 2.

35. The method of any of embodiments 1-26, wherein the antibody, or antigen binding fragment thereof, comprises a light chain comprising three CDRs chosen from the CDRs shown in Table 3.

36. The method of any of embodiments 1-26, wherein the antibody, or antigen binding fragment thereof, comprises a heavy chain comprising three CDRs chosen from the CDRs shown in Table 2 and a light chain comprising three CDRs chosen from the CDRs shown in Table 3.

37. The method of any of embodiments 34-36, wherein the CDRs comprise the CDRs of mAb 7.251.3.

38. The method of any of embodiments 34-37, wherein the CDRs comprise the CDRs of mAb 7.34.1.

39. The method of any of embodiments 34-38, wherein the CDRs comprise the CDRs of mAb 7.159.2.

40. The method of any of embodiments 1-38, wherein said patient is a human.

41. A method of neutralizing IGF-I and IGF-II in a patient, said method comprising administering to the patient at least two doses of an antibody, or antigen binding fragment thereof, which binds IGF-I and IGF-II, wherein doses are separated by about a week, and wherein each dose is between about 1.5 mg per kg of body mass and about 15 mg per mg per kg of body mass.

42. The method of embodiment 40, wherein the administering comprises administering at least three of said doses for three weeks.

43. A method of neutralizing IGF-I and IGF-II in a patient, said method comprising administering to the patient at least two doses of an antibody, or antigen binding fragment thereof, which binds IGF-I and IGF-II, wherein doses are separated by about three weeks, and wherein each dose is between about 30 mg per kg of body mass and about 45 mg per mg per kg of body mass.

44. The method of any of embodiments 41-43, wherein each dose is sufficient to neutralize IGF-I by greater than about 40% and IGF-II by greater than about 29%.

45. The method of any of embodiments 41-43, wherein each dose is sufficient to neutralize IGF-I and IGF-II by greater than about 90%.

46. The method of any of embodiments 41-45, wherein the dose is sufficient to neutralize IGF-I and IGF-II for at least one day.

47. The method of any of embodiments 41-45, wherein each dose is sufficient to neutralize IGF-I and IGF-II for least about one week.

48. The method of any of embodiments 41-45, wherein each dose is sufficient to neutralize IGF-I and IGF-II for at least about three weeks.

49. The method of any of embodiments 41-48, wherein IGF-I and IGF-II are neutralized in the blood of the patient.

50. The method of any of embodiments 41-48, wherein IGF-I and IGF-II are neutralized in a tumor of the patient.

51. The method any of embodiments 41, 42, and 44-50, wherein each said dose comprises about 1.5 mg per kg of body mass to about 15 mg per kg of body mass.

52. The method any of embodiments 41, 42, and 44-50, wherein each said dose comprises about 5 mg per kg of body mass.

53. The method any of embodiments 41, 42, and 44-50, wherein each said dose comprises about 10 mg per kg of body mass.

54. The method any of embodiments 40, 41, and 44-50, wherein each said dose comprises about 15 mg per kg of body mass.

55. The method of any of embodiments 43-50, wherein each dose comprises about 30 mg per kg of body mass.

56. The method of any of embodiments 43-50, wherein each dose comprises about 45 mg per kg of body mass.

57. The method of any of embodiments 41-56, wherein the administering comprises administering one or more loading doses followed by one or more maintenance doses, and wherein said loading doses are at least about two times greater than said maintenance doses.

58. The method of any of embodiments 41-56, wherein the patient suffers from a cancer of the breast, bladder, prostate, colon, uterus, rectum, throat, lung, a colorectal cancer, non-small cell lung cancer, a sarcoma, or hepatocellular carcinoma.

59. The method of embodiment 58, wherein the cancer is bladder cancer.

60. The method of embodiment 58, wherein the cancer is hepatocellular carcinoma.

61. The method of embodiment 58, wherein the cancer is breast cancer.

62. The method of embodiment 58, wherein the cancer is a sarcoma.

63. The method of embodiment 58, wherein the cancer is prostate cancer.

64. The method of embodiment 58, wherein the cancer is rectal cancer.

65. The method of any of embodiments 58-64, wherein the cancer is a primary tumor cancer.

66. The method of any of embodiments 58-64, wherein the tumor cancer is a metastatic tumor cancer.

67. The method of any of embodiments 41-66, wherein the antibody which binds IGF-I and IGF-II is selected from among mAb 7.251.3, mAb 7.34.1, and mAb 7.159.2.

68. The method of embodiment 67, wherein the antibody which binds IGF-I and IGF-II is mAb 7.251.3.

69. The method of embodiment 67, wherein the antibody which binds IGF-I and IGF-II is mAb 7.34.1.

70. The method of embodiment 67, wherein the antibody which binds IGF-I and IGF-II is mAb 7.159.2.

71. The method of any of embodiments 41-66, wherein the antibody, or antigen binding fragment thereof, comprises at least one variable chain polypeptide having an amino acid sequence chosen from SEQ ID NOs: 2, 6, 10, 14, 18, 40, 44, 48, 52, 56, 60, 64, 68, and 72.

72. The method of any of embodiments 41-66, wherein the antibody, or antigen binding fragment thereof, comprises at least one variable chain polypeptide having an amino acid sequence chosen from SEQ ID NOs: 4, 8, 12, 16, 20, 42, 46, 50, 54, 58, 62, 66, 70, 74.

73. The method of any of embodiments 41-66, wherein the antibody, or antigen binding fragment thereof, comprises at least one variable chain polypeptide having an amino acid sequence chosen from SEQ ID NOs: 2, 6, 10, 14, 18, 40, 44, 48, 52, 56, 60, 64, 68, and 72, and at least one variable chain polypeptide having an amino acid sequence chosen from SEQ ID NOs: 4, 8, 12, 16, 20, 42, 46, 50, 54, 58, 62, 66, 70, 74.

74. The method of any of embodiments 41-66, wherein the antibody, or antigen binding fragment thereof, comprises a heavy chain comprising three CDRs chosen from the CDRs shown in Table 2.

75. The method of any of embodiments 41-66, wherein the antibody, or antigen binding fragment thereof, comprises a light chain comprising three CDRs chosen from the CDRs shown in Table 3.

76. The method of any of embodiments 41-66, wherein the antibody, or antigen binding fragment thereof, comprises a heavy chain comprising three CDRs chosen from the CDRs shown in Table 2 and a light chain comprising three CDRs chosen from the CDRs shown in Table 3.

77. The method of any of embodiments 74-76, wherein the CDRs comprise the CDRs of mAb 7.251.3.

78. The method of any of embodiments 74-77, wherein the CDRs comprise the CDRs of mAb 7.34.1.

79. The method of any of embodiments 74-78, wherein the CDRs comprise the CDRs of mAb 7.159.2.

80. The method of any of embodiments 41-79, wherein the patient is a human.

81. The method of any of embodiments 1-81, wherein the antibody, or antigen binding fragment thereof, binds to IGF-II with greater affinity than to IGF-I.

82. The method of embodiment 81 wherein, the antibody, or antibody fragment thereof binds to IGF-II with an affinity greater than the affinity for IGF-I chosen from, at least 2.5 at least 5, at least 10, at least 25, at least 50 or at least 150 times greater affinity for IGF-II than for IGF-I.

83. The method of any of embodiments 1-82, wherein the antibody, or antigen binding fragment thereof, is administered at a dose providing a Cmax from about 70 to about 588 μg/ml.

84. The method of any embodiments 1-83, wherein, the antibody, or antigen binding fragment thereof, is administered at a dose providing an AUC from about 90 to about 3620 μg*d/ml.

85. The method of any of embodiments 1-82, wherein the antibody, or antigen binding fragment thereof, is administered at a dose providing a Cmax of about 70 μg/ml.

86. The method of any of embodiments 1-82, wherein the antibody, or antigen binding fragment thereof, is administered at a dose providing a Cmax of about 170 μg/ml.

87. The method of any of embodiments 1-82, wherein the antibody, or antigen binding fragment thereof, is administered at a dose providing a Cmax of about 260 μg/ml.

88. The method of any of embodiments 1-82, wherein the antibody, or antigen binding fragment thereof, is administered at a dose providing a Cmax of about 560 μg/ml.

89. The method of any of embodiments 1-82, wherein the antibody, or antigen binding fragment thereof, is administered at a dose providing a Cmax of about 461 μg/ml.

90. The method of any of embodiments 1-82, wherein the antibody, or antigen binding fragment thereof, is administered at a dose providing a Cmax of about 588 μg/ml.

91. The method of any embodiments 1-83, wherein, the antibody, or antigen binding fragment thereof, is administered at a dose providing an AUC of about 90 μg*d/ml.

92. The method of any embodiments 1-83, wherein, the antibody, or antigen binding fragment thereof, is administered at a dose providing an AUC of about 415 μg*d/ml.

93. The method of any embodiments 1-83, wherein, the antibody, or antigen binding fragment thereof, is administered at a dose providing an AUC of about 600 μg*d/ml.

94. The method of any embodiments 1-83, wherein, the antibody, or antigen binding fragment thereof, is administered at a dose providing an AUC of about 1940 μg*d/ml.

95. The method of any embodiments 1-83, wherein, the antibody, or antigen binding fragment thereof, is administered at a dose providing an AUC of about 1320 μg*d/ml.

96. The method of any embodiments 1-83, wherein, the antibody, or antigen binding fragment thereof, is administered at a dose providing an AUC of about 3620 μg*d/ml.

### EXAMPLES

While the disclosure has been provided in detail with reference to particular examples thereof, it will be apparent to one skilled in the art that various changes can be made, and equivalents employed, without departing from the scope of the disclosure. The following examples are further provided solely for the purpose of illustrating aspects of the methods described herein and should not be taken as limiting the disclosure in any way.

### Example 1

### MEDI-573

MEDI-573 is a fully human immunoglobulin G2 lambda (IgG2) antibody generated with Xenomouse® technology and manufactured in Chinese Hamster Ovary (CHO) cells. MEDI-573 selectively binds to human insulin-like growth factors hIGF-I and hIGF-II and inhibits insulin-like growth factor IGF-I and IGF-II mediated signal transduction in tumor cells, thereby inhibiting tumor growth. The antibody was isolated from mice immunized alternately with soluble recombinant human hIGF-I and hIGF-II coupled to keyhole limpet hemocyanin (KLH), as described in U.S. Pat. No. 7,939,637. MEDI-573 is composed of 2 light chains and 2 heavy chains, with an overall molecular weight of approximately 151 kilodaltons.

### Example 2

### Pharmacology in Mice

To determine the effects of MEDI-573 on the in vivo growth of cells expressing IGF-1R and either IGF-I or IGF-II ligands, antitumor efficacy studies were performed. P12 (NIH3T3 cells ectopically overexpressing human IGF-I and human IGF-1R) or C32 (NIH3T3 cells ectopically overexpressing human IGF-II and human IGF-1R), were implanted into the right flank of female athymic nude mice. Mice were randomized 14 (P12) or 17 (C32) days post implant when tumors reached 110 and 175 mm³ in volume, respectively. For these studies, MEDI-573 was administered intraperitoneally (IP) at doses of 0, 3, 10, 30, and 60 mg/kg, twice per week, for a total of 4 doses. MEDI-573 significantly inhibited the growth of P12 tumors. A clear dose response was observed between the dose levels of 3 to 30 mg/kg in which, 3, 10, and 30 mg/kg resulted in tumor growth inhibition (TGI) of 20%, 66%, and 86%, respectively. When dosed higher at 60 mg/kg, the resulting TGI was similar to that of 30 mg/kg. Similarly, MEDI-573 was highly efficacious against the C32 model, when administered as a single agent with a maximal TGI of 91% at the highest dose administered. An antitumor dose response was observed between the dose levels of 3 to 30 mg/kg ranging from 18% TGI to 86% TGI.

Phosphorylation of IGF-1R was examined in both the P12 and C32 tumor models. Tumors were harvested at 24 or 72 hours following the last dose of mAb and analyzed for changes in phosphorylation levels using the Insulin Signaling Panel (Total Protein) and Insulin Signaling Panel (Phosphoprotein) Whole Cell Lysate kits from Meso Scale Discovery® (MSD), following the manufacturer's instructions. Phospho-IGF-1R signals were normalized to total-IGF-1R signals and plotted against the dosing of each group. For the P12 model at the 24-hour time point, marked inhibition of phosphorylated insulin-like growth factor 1 receptor (pIGF-1R) was observed at doses greater than 3 mg/kg. Interestingly however, at the 72-hour time point, some recovery of the pIGF-1R was observed in the 10 and 30 mg/kg groups. At the highest dose examined, 60 mg/kg, the suppression of pIGF-1R was maintained at the 72-hour time point. In comparison, there was a dose-dependent inhibition of pIGF-1R and the observed reduction was maintained up to the 72-hour time point in the C32 model. The observed difference between P12 and C32 in the recovery of pIGF-1R may be attributed to the differences in affinity of MEDI-573 to IGF-II and IGF-I. The results showed that MEDI-573 inhibited the growth of both C32 and P12 in a dose-dependent manner in tumor models in vivo. Further, MEDI-573 caused significant reduction in the phosphorylation levels of IGF-1R in both P12 and C32 models.

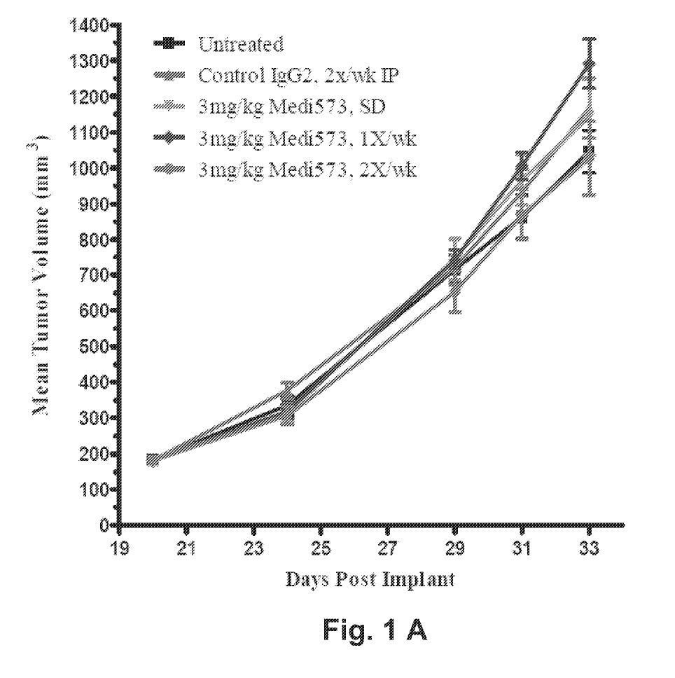

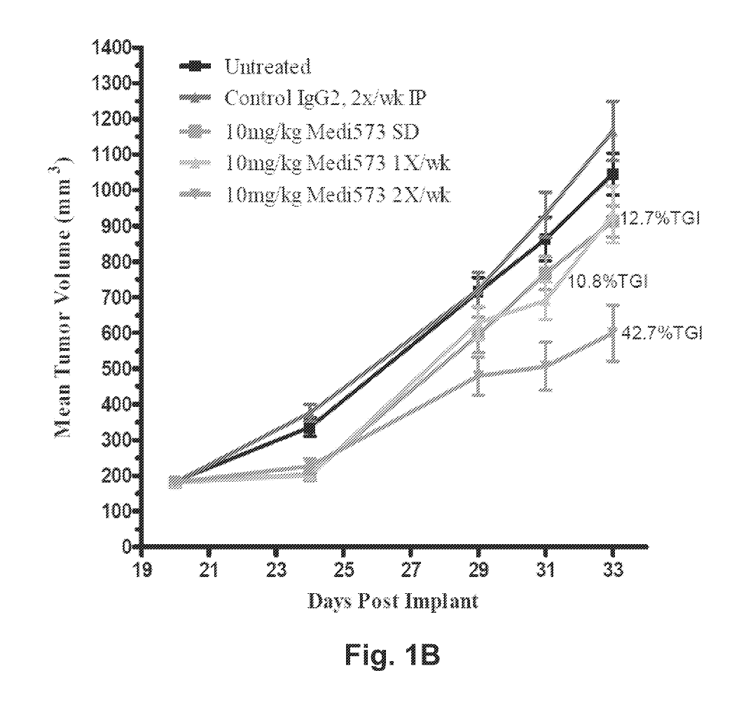

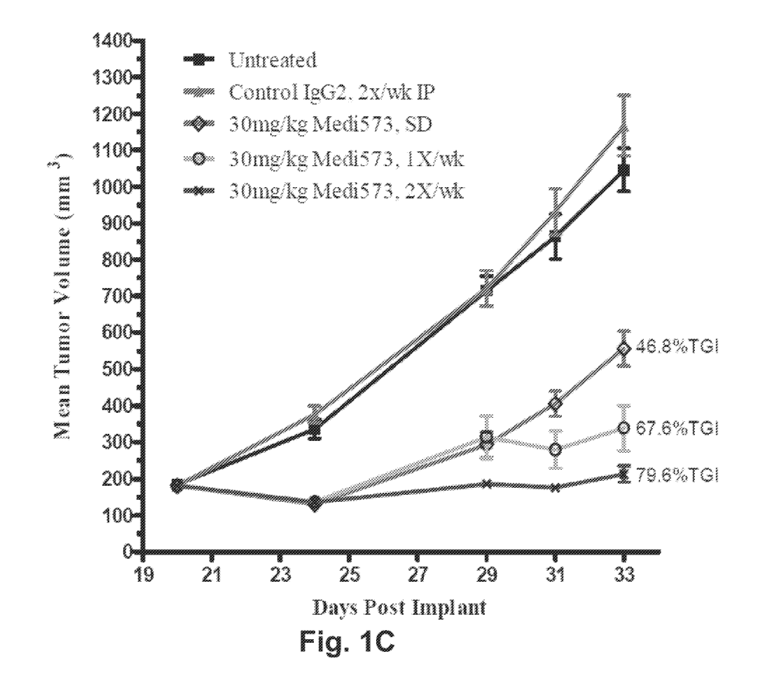

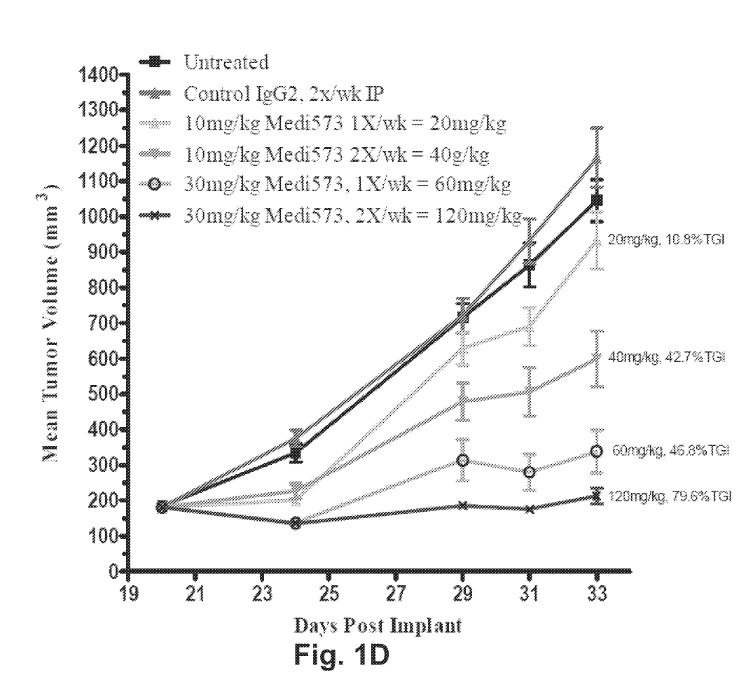

The in vivo antitumor activity of MEDI-573 was tested at 3 different doses and administration schedules in athymic nude mice implanted with C32 cells (NIH cells that ectopically overexpress human IGF-II and human IGF-1R). MEDI-573 was administered IP at 3, 10, and 30 mg/kg according to 3 dosing schedules (single dose, 1 injection per week [once per week], and 2 injections per week [twice per week]) for each dose tested. When administered at 3 mg/kg, MEDI-573 failed to inhibit C32 tumor growth regardless of dosing schedule. At the 10 mg/kg dose, significant (48%) TGI was observed when MEDI-573 was administered twice per week. At the highest dose of 30 mg/kg, TGI was clearly schedule dependent with better efficacy demonstrated when MEDI-573 was administered twice per week for 2 weeks than once per week for 2 weeks, which in comparison was better than a single dose. MEDI-573 significantly inhibited C32 tumor growth in vivo. Clear accumulative (or total) dose dependent TGI was observed, indicating that maintaining high levels of MEDI-573 suppresses tumor growth. (FIG. 1) MEDI-573 can be maintained at high level by, for example, increasing the frequency of lower doses or administering higher doses less frequently.

### Example 3

### Pharmacokinetics in Cynomulgus Monkeys

The objective of this GLP study was to evaluate toxicity and toxicokinetics in cynomolgus monkeys following a once-weekly IV administration of MEDI-573 for 13 weeks and to follow the recovery from any potential toxic effects during an 8-week treatment-free period. In this study, the PK properties of both free and total MEDI-573 were evaluated following administration of the antibody during study Days 1 to 8. Additionally, steady-state serum concentrations of MEDI-573 were evaluated following administration of the antibody once-weekly via a 30-minute IV infusion for 13 weeks. Based on the results obtained in this study, the PK properties of MEDI-573 (total antibody) following multiple-dose administration were both linear (dose-independent) and stationary (time-independent).

Determination of concentrations of MEDI-573 in serum was performed using a qualified antigen capture assay based on the Meso Scale Discovery® (MSD) detection platform for evaluation of free antibody. The method utilized recombinant human insulin-like growth factor-II (IGF-II) as a capture reagent and ruthenium-labeled goat F(ab′)2 anti-human IgG (γchain specific) as the detection reagent. The working range of the assay was 9.77 to 625 ng/ml in 10% cynomolgus monkey serum matrix with a sensitivity of 97.7 ng/ml in 100% matrix. This method quantified the concentration of free antibody (i.e., antibody not bound to the IGF-I or IGF-II antigens in the specified matrix).

MEDI-573 concentration data were analyzed by non-compartmental analysis (NCA). NCA analysis was performed using WinNonlin Professional (version 5.2, Pharsight Corp., Mountain View, Calif.). A summary of the PK parameter estimates is provided in Table 4.

TABLE 4
Summary of PK Estimates for MEDI-573 in Cynomulgous Monkeys

| | | | | | AUC_{∞}/Dose |
|---|---|---|---|---|---|
| Dose (mg/kg) | Cₘₐₓ | Vₛₛ | CL | AUC_{∞} | (μg × day/mL)/ |
| (Group, N) | (μg/mL) | (mL/kg) | (mL/day/kg) | (μg × day/mL) | (μg/kg) |
| 1 | 33.9 ± 25.6 | 32.6 ± 22.1 | 16.4 ± 9.78 | 76.1 ± 34.2 | 0.076 ± 0.034 |
| (2, 6) | | | | | |
| 10 | 176 ± 16.2 | 57.3 ± 9.94 | 17.1 ± 1.76 | 588 ± 61.1 | 0.059 ± 0.006 |
| (3, 6) | | | | | |
| 60 | 1343 ± 409 | 57.8 ± 8.83 | 12.6 ± 3.15 | 5102 ± 1670 | 0.085 ± 0.028 |
| (4, 10) | | | | | |

Values presented as mean±SD. Cmax=maximum serum concentration, Vss=volume of distribution at steady state, CL=clearance, AUC=area under the curve.

Additionally, the mean PK profiles from all dose groups in this study were modeled simultaneously using a one-compartment mammillary PK model. The PK model provided a good description of the experimental data. The estimated mean clearance for MEDI-573 following the 1, 10, and 60 mg/kg dose using the PK modeling approach was 14.7 mL/day/kg, 17.2 mL/day/kg, and 11.8 mL/day/kg, respectively. Pharmacokinetic properties of MEDI-573 following multiple-dose administration highlighted the time-independent nature of MEDI-573 PK. During Weeks 2 to 12, dose-dependent increases in steady-state serum concentration of MEDI-573 (data mean±standard deviation [SD]) were observed following the administration of weekly doses of the antibody. The mean steady-state serum concentrations (free antibody) at 1, 10, and 60 mg/kg for each of the dosing intervals (maximum concentration at steady state [CSS max]) were 19.2±1.68, 266±22.4, and 2174±194 μg/mL, respectively.

### Example 4

### Pharmacokinetic/Pharmacodynamic Study of MEDI-573 in Cynomulgus Monkeys

This non-GLP study was designed to evaluate the PK and PD properties of MEDI-573 in male cynomolgus monkeys following intravenous administration of the mAb on Day 1 and Day 8. Four animals were randomly assigned to 4 treatment groups: Group 1 (n=1, 1 mg/kg), Group 2 (n=1, 3 mg/kg), Group 3 (n=1, 10 mg/kg) and Group 4 (n=1, 30 mg/kg). Samples (serum for PK and plasma for biomarker analysis) were collected at various pre-assigned time points throughout the study. Determination of concentrations of MEDI-573 in serum was performed using a qualified antigen capture assay based on the Meso Scale Discovery® (MSD) detection platform for evaluation of free antibody. The same methodology as described in Example 3 was used to measure MEDI-573 concentrations.

Determination of changes in the biomarker profiles (free IGF-I and IGF-II) following administration of MEDI-573 on Days 1 and 8 was performed using qualified analytical procedures. The concentrations of free IGF-I and IGF-II in plasma were determined using biotinylated MEDI-573 as a capture reagent and either a ruthenium-labeled polyclonal antibody directed against IGF-I (for detection of free IGF-I) or IGF-II (for detection of free IGF-II) as the detection reagents. A blocked MSD Standard Bind Streptavidin plate was coated with 2 μg/ml of the capture reagent and incubated for at least 30 minutes at room temperature. The analyte solutions were then loaded into the wells of the coated plate to allow a capture duration of 10 minutes at ambient temperature with gentle agitation. The assay plate was subsequently washed before the addition of the detection reagent to allow a detection duration of 30 minutes at ambient temperature with gentle agitation, followed by another wash before the addition of the substrate (1× Read Buffer T), and then read using the MSD Sector Imager 2400 (Meso Scale Discovery). The limit of detection for each assay was: Free IGF-I assay=0.313 ng/mL; Free IGF-II assay=0.625 ng/mL.

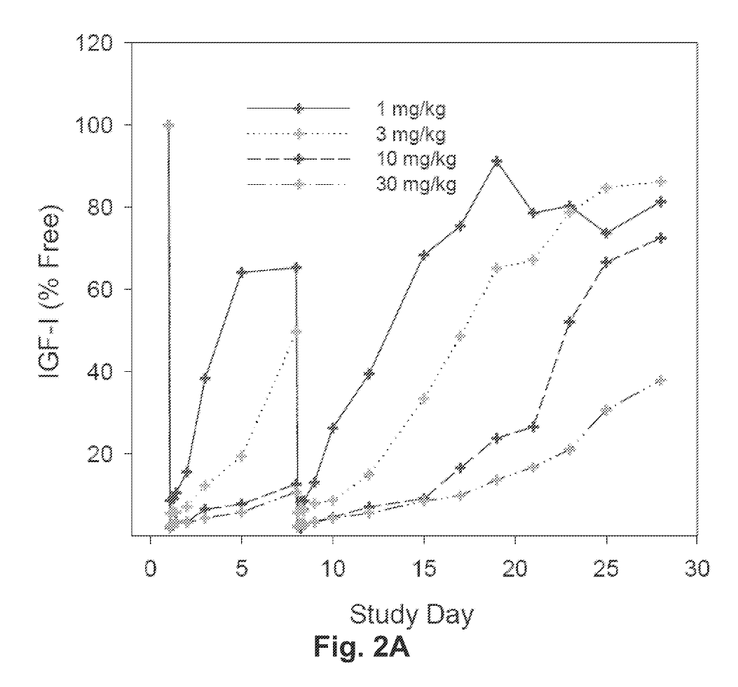

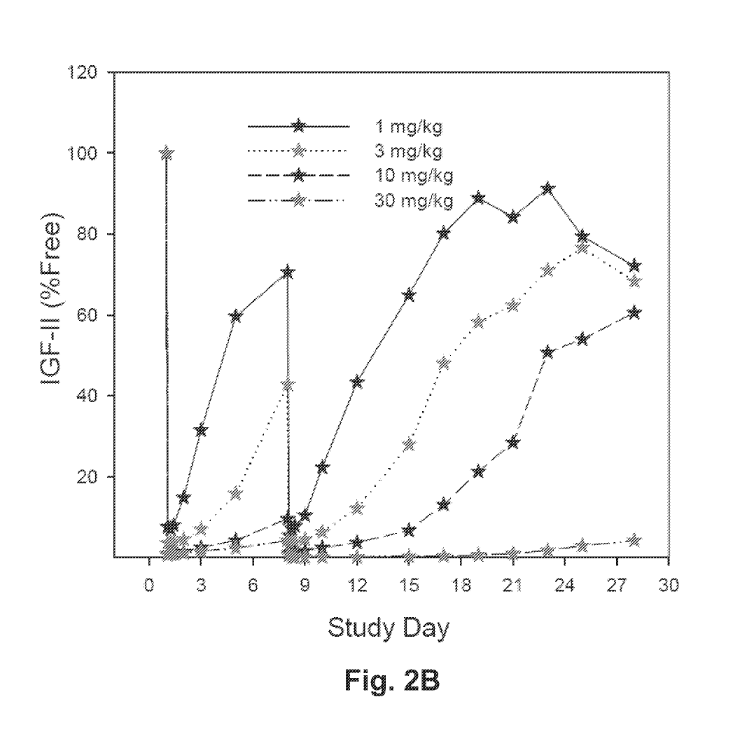

Any changes in the concentrations of free IGF-I and IGF-II post antibody administration were normalized to baseline concentrations of the antigens determined in the samples collected prior to administration of MEDI-573 (predose samples) in each animal, and were expressed as percent change from the baseline. Following administration of MEDI-573, dose-dependent changes in free IGF-I and IGF-II were observed. (FIG. 2)

### Example 5

### A 13-week, Intravenous, Toxicity, and Toxicokinetic Study with Recovery of MEDI-573 in Cynomolgus Monkeys

A GLP, 13-week, repeat dose, IV infusion, toxicity study was conducted in cynomolgus monkeys using MEDI-573 at 1, 10, or 60 mg/kg/dose, with a dose volume of 6 mL/kg/dose. Control animals were administered 6 mL/kg/dose of vehicle (saline, 0.9% sodium chloride for injection). MEDI-573 was administered by a 30-minute continuous IV infusion, once weekly for a total of 13 doses.

Following administration of MEDI-573 to the cynomolgus monkeys, dose-dependent decreases in the serum concentrations of both IGF-I and IGF-II were observed. Relative to baseline IGF concentrations (Day 1, predose), the average suppression of free IGF-I in animals administered 10 mg/kg MEDI-573 was greater than 95% from study Weeks 2 to 12. The IGF-II concentrations in the 10 mg/kg dose group were BLQ (0.08 ng/mL). From study Week 2 to Week 12, serum concentrations of free IGF-I and IGF-II were BLQ in animals administered 60 mg/kg MEDI-573. The NOAEL was 60 mg/kg/week, which resulted in a dose normalized AUC (AUC∞/Dose) of 0.085±0.028 (μg˜day/mL)/(μg/kg), a mean clearance of 12.6±3.15 mL/day/kg, and a mean Cmax value of 1343±409 μg/mL.

In summary, MEDI-573 was well tolerated following up to 13, once-weekly, 30-minute continuous IV infusion administrations in cynomolgus monkeys (the relevant toxicology species) with a NOAEL of 60 mg/kg, the highest dose tested. MEDI-573 was fully pharmacologically active with plasma concentrations at or above the concentration needed for full suppression of serum IGF-I and IGF-II.

### Example 6

### Pharmacokinetics, Pharmacodynamics and Activity in Humans

A Phase 1, multicenter, open-label, single-arm, dose-escalation and dose-expansion study has been conducted to evaluate the safety, tolerability, and antitumor activity of MEDI-573 in adult human subjects with advanced solid tumors refractory to standard therapy or for which no standard therapy exists. Cohorts of evaluable subjects at multiple sites each received one of five dosage levels of MEDI-573 (0.5, 1.5, 5, 10 or 15 mg per kg) every 7 days. The dosing cohort is shown in Table 5.

TABLE 5
Dosing Cohort

| MEDI-573 Dosing | |
|---|---|
| Cohort (mg/kg) | No. of Subject (N = 18) |
| 0.5 | 4 |
| 1.5 | 3 |
| 5 | 4 |
| 10 | 3 |
| 15 | 4 |

MEDI-573 (110 mg/vial) was formulated as lyophilized powder stored at 2°-8° C. for reconstitution in 4 ml water containing polysorbate-80, trehalose dehydrate, L-histidine, and L-histidine hydrochloride monohydrate. MEDI-573 was administered on Days 1, 8, and 15 of each 21-day treatment cycle) as a 60-minute intravenous (IV) infusion until unacceptable toxicity, documentation of disease progression, or other reasons for subject withdrawal. Intra-subject dose escalation was not allowed. Dose escalation followed a standard 3+3 study design.

Eighteen patients (7M/11F, median age 58 yrs) were treated across weekly dose levels of 0.5, 1.5, 5, 10 or 15 mg per kg as summarized in Table 6.

TABLE 6
Population Demographics.

| Characteristics | (n = 18) |
|---|---|
| Age, years | |
| Median | 58 |
| Range | 37-78 |
| Performance status | |
| Median | 80 |
| Range | 70-100 |
| Sex | |
| Male | 7 |
| Female | 11 |
| No. of prior therapeutic regimens: | |
| Median | 5 |
| Range | 1-9 |
| Tumor type | |
| Soft tissue sarcoma | 3 |
| Prostate | 3 |
| Pancreatic | 2 |
| Anal | 1 |
| Adenocarcinoma | 1 |
| Adrenocortical | 1 |
| Bladder | 1 |
| Bone sarcoma | 1 |
| Breast | 1 |
| Colon | 1 |
| Esophageal | 1 |
| Rectal | 1 |
| Uterine | 1 |

Blood samples for assessment of PK parameters of MEDI-573 and anti-MEDI-573 antibodies were collected. At Cycle 1 Day 1, serum for PK analysis was collected immediately prior to infusion, immediately following infusion, and at 2 and 6 hours post infusion. Additional PK samples were collected on Day 2 at 24 hours post Day 1 infusion ±2 hours, Day 3 at 48 hours post Day 1 infusion ±2 hours, Day 8 and Day 15 pre-infusion and immediately after the infusion. Subsequently PK samples were collected pre-infusion and immediately following infusion (±5 minutes) of MEDI-573 every 7 days (i.e., on Days 1, 8, and 15 of each cycle), at the time of discontinuation of MEDI-573, and at 21 and 30 days post-therapy and 3 months post-therapy. Samples for anti-MEDI-573 antibodies were collected at Screening and subsequently prior to each infusion of MEDI-573.

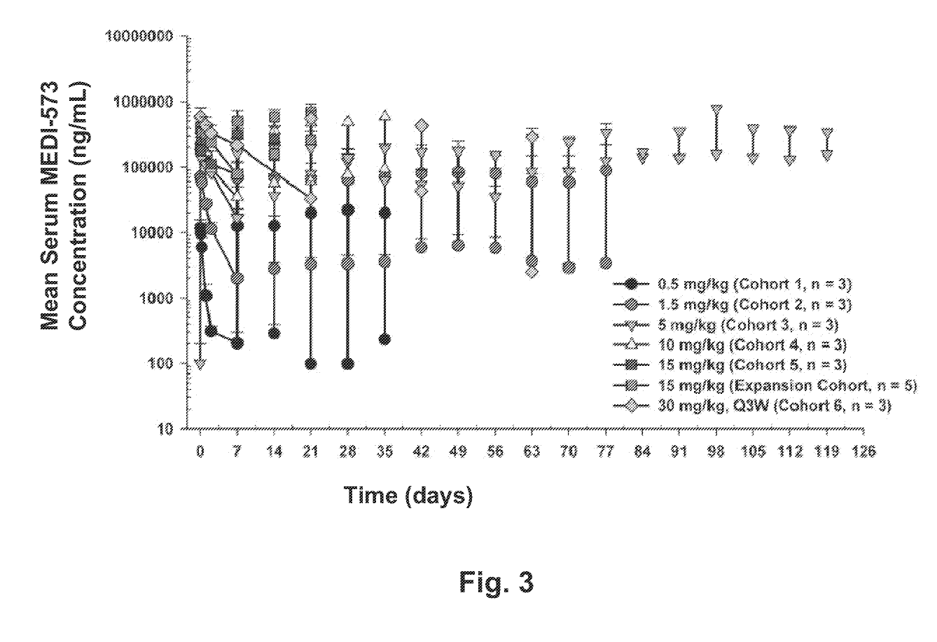

FIG. 3 shows pharmacokinetic results at time points through the study. MEDI-573 exhibited a dose-proportional increase in exposure, with an AUC of 415±165, 597±298, and 1940±904 mcg*d/mL at 5, 10 and 15 mg per kg, respectively. See Table 7.

TABLE 7
Pharmacokinetic results of MEDI-573 in Adult Humans

| | | | T_{MAX} | |
|---|---|---|---|---|
| Dose (mg/kg) | Subjects | C_{MAX} (μg/mL) | (days) | AUC_{τ} (μg * d/mL) |
| 0.5 | 3 | 11.7 | 0.0417 | 6.10 |
| 1.5 | 3 | 71.8 | 0.0417 | 92.1 |
| 5 | 3 | 172 | 0.125 | 415 |
| 10 | 3 | 263 | 0.0695 | 597 |
| 15 | 3 | 560 | 0.236 | 1940 |

C_{MAX} = peak concentration;
T_{MAX} = Time to peak concentration;
AUC = area under the curve

The method used to determine the concentrations of MEDI-573 in human serum utilized a biotinylated monoclonal anti-idiotype antibody as a capture reagent and ruthenium-labeled monoclonal anti-idiotype antibody as the detection reagent. The assay was a solution-phase bridging assay which utilized the biotinylated and ruthenylated anti-idiotype antibodies to form a bridging complex with MEDI-573 in order to generate an ECL signal and detect the presence of MEDI-573. The serum samples were initially incubated with an equal volume of the biotinylated and ruthenium-labeled anti-idiotype antibody solution for 1 hour (±10 minutes), after which 25 μL the solution mixtures were loaded onto the wells of the blocked MSD Standard Bind streptavidin plates and incubated for 30 minutes at ambient temperature with gentle shaking. The assay plates were then washed before addition of the Read Buffer T (1×) solution and read using the MSD Sector Imager. MEDI-573 concentration data were analyzed by non-compartmental analysis (NCA). NCA analysis was performed using WinNonlin Professional (version 5.2, Pharsight Corp., Mountain View, Calif.).

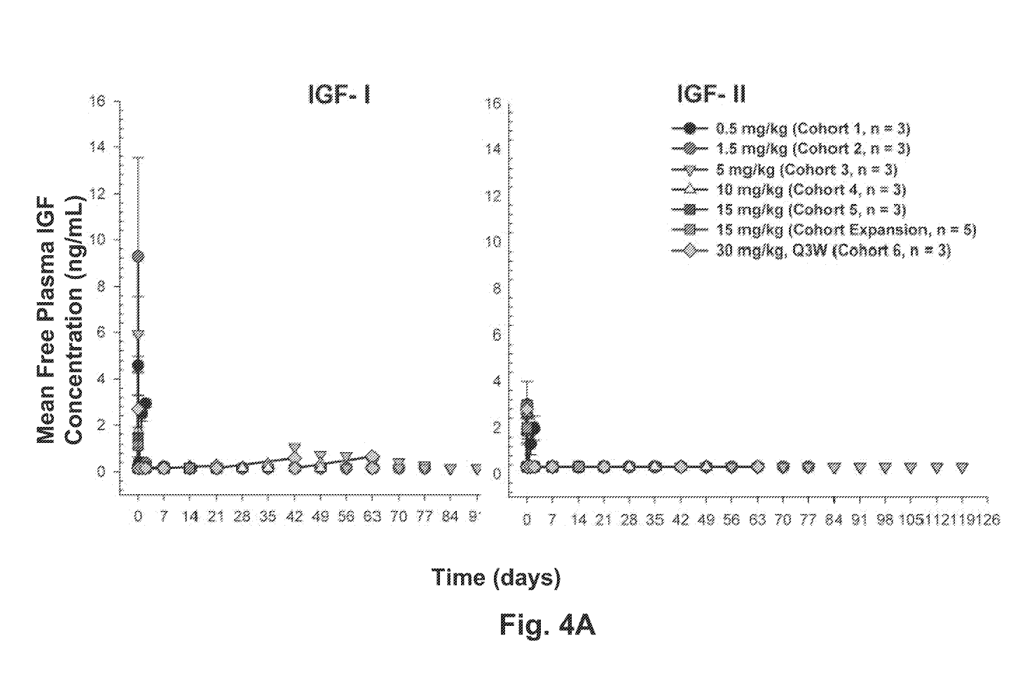

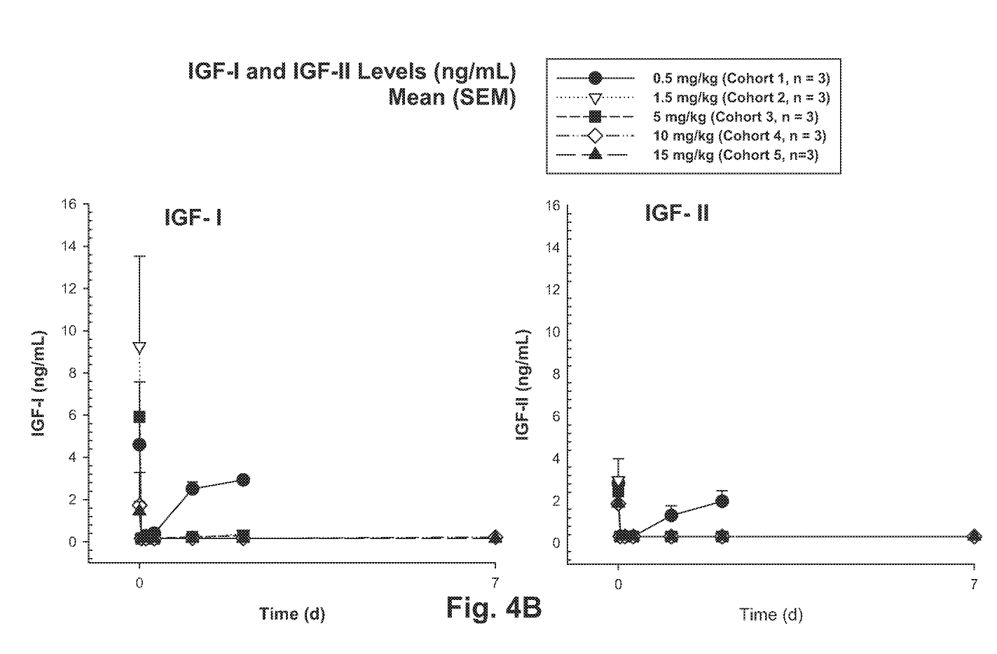

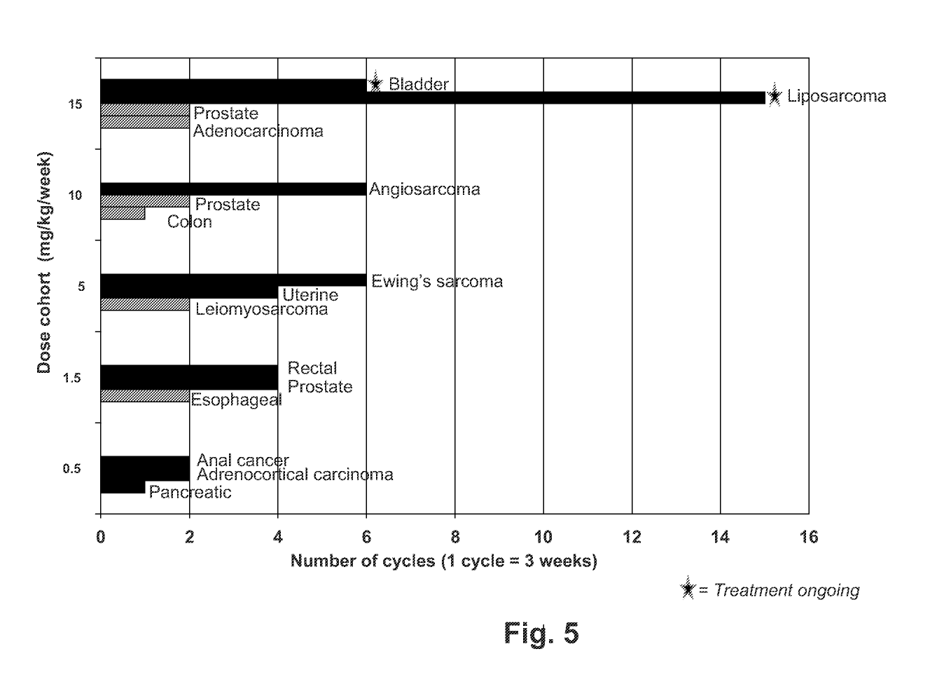

FIG. 4 shows the levels of IGF-I and IGF-II in the patient's circulation at time points following administration of MEDI-573. IGF-II was fully suppressed at 5, 10, and 15 mg per kg for the duration of the study. IGF-I was also fully suppressed at 5, 10 and 15 mg per kg, for the duration of the study, with the exception of one subject in the 5 mg per kg group who showed less than complete, but still greater than 90%, suppression.

At 5 mg per kg, IGF-I and IGF-II were suppressed below the limit of detection at seven days after administration. FIG. 4B. At 0.5 mg per kg, both IGF-I and IGF-II were initially suppressed to a level below the limit of quantitation. But by day 2, both IGF-I and IGF-II had increased. FIG. 4B. At day 2 for 0.5 mg per kg, IGF-1 rose to 2.9 ng/ml. Compared to baseline at 4.9 ng/ml in that group, IGF-I suppression was about 40%. IGF-II rose to 2.0 ng/ml. Compared to baseline at 2.8 ng/ml, IGF-II suppression was about 29%. This indicates that a dose of 0.5 mg per kg can suppress IGF-I and IGF-II initially, but cannot maintain that suppression in a once per week dosing regimen.

Seven of 16 subjects showed disease stabilization with two patients continued on treatment. Specifically, subjects suffering from bladder cancer, liposarcoma, angiosarcoma, Ewing's sarcoma, uterine cancer, rectal cancer, and prostate cancer showed disease stabilization. Two subjects have remained on study treatment (bladder cancer, 6+ cycles; liposarcoma, 15+ cycles). The majority of patients showing disease stabilization were treated with 5 mg per kg or greater, indicating that the at least 90% suppression observed with this dosing regimen is sufficient for therapeutic benefit. Conversely, none of three patients dosed with 0.5 mg per kg showed disease stabilization, consistent with the observation that this dose fails to maintain the initial level of suppression of IGF-I and IGF-II for more than one day, and does not maintain the suppression observed at higher doses.

The combination of PD, PK, and activity data indicate that suppression of IGF-I and IGF-II should be greater than about 40% and 29%, respectively, for therapeutic benefit. Moreover, these data strongly support a dosing regimen that maintains suppression of at least 90% of IGF-I and IGF-II during the course of treatment, which can be achieved with a dose of at least about 5 mg per kg administered about every week.

No DLTs or drug-related serious adverse events have been reported to date. (NCI CTC AE V3.0) No significant changes to plasma glucose or insulin levels have been reported and no serious toxicity patterns have been noted. Although MEDI-573 has not been shown to alter glycemic control in nondiabetic patients, its metabolic effect in diabetics is not yet known because patients with diabetes were excluded from the study.

MEDI-573 is well tolerated and has a favorable PK profile. Consistent with its lack of affinity for insulin, MEDI-573 does not affect insulin-mediated glucose metabolism at the doses tested, which have all been administered to nondiabetic patients. Antitumor activity has been suggested by disease stabilization of greater than 3 months in multiply refractory patients with several different solid tumor types.

What is claimed is:

1. A method of treating cancer in a patient, said method comprising administering to the patient an antibody, or antigen-binding fragment thereof, which binds Insulin Growth Factor-I (IGF-I) and Insulin Growth Factor-II (IGF-II), wherein the treatment comprises:
(a) administering to the patient at least two doses of the antibody or antigen-binding fragment thereof, wherein doses are separated by about a week, and wherein each dose is between about 1.5 mg per kg of body mass and about 15 mg per kg of body mass or
(b) administering to the patient at least two doses of the antibody, or antigen binding fragment thereof, wherein doses are separated by about three weeks, and wherein each dose is between about 30 mg per kg of body mass and about 45 mg per kg of body mass.

2. The method of claim 1(a), wherein the administering comprises administering at least three of said doses for about three weeks.

3. (canceled)

4. The method of claim 1, wherein each dose is sufficient to neutralize IGF-I and IGF-II for at least one day.

5. The method of claim 1, wherein each dose is sufficient to neutralize IGF-I and IGF-II for at least about one week.

6. The method of claim 1(b), wherein each dose is sufficient to neutralize IGF-I and IGF-II for at least about three weeks.

7. The method of claim 1, wherein each dose is sufficient to neutralize IGF-I by greater than about 40% and IGF-II by greater than about 29% in samples from treated subjects relative to biological samples from untreated subjects.

8-16. (canceled)

17. The method of claim 1, wherein the administering comprises administering one or more loading doses followed by administering one or more maintenance doses, and wherein said loading doses are at least about two times greater than said maintenance doses.

18. The method of claim 1, wherein the cancer is a cancer of the breast, bladder, prostate, colon, uterus, rectum, throat, lung, a colorectal cancer, non-small cell lung cancer, a sarcoma, or hepatocellular carcinoma.

19-25. (canceled)

26. The method of claim 1, wherein the tumor cancer is a metastatic tumor cancer.

27. The method of claim 1, wherein the antibody which binds IGF-I and IGF-II is selected from mAb 7.251.3, mAb 7.34.1, and mAb 7.159.2.

28-30. (canceled)

31. The method of claim 1, wherein the antibody, or antigen-binding fragment thereof, comprises at least one variable chain polypeptide having an amino acid sequence chosen from SEQ ID NOs: 2, 6, 10, 14, 18, 40, 44, 48, 52, 56, 60, 64, 68, and 72.

32. The method of claim 1, wherein the antibody, or antigen-binding fragment thereof, comprises at least one variable chain polypeptide having an amino acid sequence chosen from SEQ ID NOs: 4, 8, 12, 16, 20, 42, 46, 50, 54, 58, 62, 66, 70, 74.

33. (canceled)

34. The method of claim 1, wherein the antibody, or antigen-binding fragment thereof, comprises a heavy chain comprising three CDRs chosen from the CDRs shown in Table 2.

35. The method of claim 1, wherein the antibody, or antigen-binding fragment thereof, comprises a light chain comprising three CDRs chosen from the CDRs shown in Table 3.

36. The method of claim 1, wherein the antibody, or antigen-binding fragment thereof, comprises a heavy chain comprising three CDRs chosen from the CDRs shown in Table 2 and a light chain comprising three CDRs chosen from the CDRs shown in Table 3.

37. The method of claim 34, wherein the CDRs comprise the CDRs of mAb 7.251.3, wherein HCDR1 is SEQ ID NO: 21, HCDR2 is SEQ ID NO: 22, HCDR3 is SEQ ID NO: 23, LCDR1 is SEQ ID NO: 24, LCDR2 is SEQ ID NO: 25 and LCDR3 is SEQ ID NO: 26.

38. The method of claim 34, wherein the CDRs comprise the CDRs of mAb 7.34.1, wherein HCDR1 is SEQ ID NO: 27, HCDR2 is SEQ ID NO: 28, HCDR3 is SEQ ID NO: 29, LCDR1 is SEQ ID NO: 30, LCDR2 is SEQ ID NO: 31 and LCDR3 is SEQ ID NO: 32.

39. The method of claim 34, wherein the CDRs comprise the CDRs of mAb 7.159.2, wherein HCDR1 is SEQ ID NO: 33, HCDR2 is SEQ ID NO: 34, HCDR3 is SEQ ID NO: 35, LCDR1 is SEQ ID NO: 36, LCDR2 is SEQ ID NO: 37 and LCDR3 is SEQ ID NO: 38.

40-84. (canceled)
